(19) United States
(12) Patent Application Publication
(10) Pub. No.: US 2024/0268976 A1
(43) Pub. Date: Aug. 15, 2024

(54) STENT SYSTEM

(71) Applicants: The Regents of the University of California, Oakland, CA (US); VS3 Medical Inc., Burlingame, CA (US)

(72) Inventors: Matthew Amans, San Francisco, CA (US); Michael J. Horzewski, San Jose, CA (US)

(21) Appl. No.: 18/441,223

(22) Filed: Feb. 14, 2024

Related U.S. Application Data
(60) Provisional application No. 63/445,771, filed on Feb. 15, 2023.

(51) Int. Cl. A61F 2/90

(52) U.S. Cl. CPC A61F 2/90; A61F 2230/0023; A61F 2250/001; A61F 2250/0018

(57) ABSTRACT

A stent assembly for implantation into a blood vessel includes an inlet zone having at least one inlet stent structure, wherein the inlet stent structure is self-expanding and has an inlet radial force configured to conform to a non-circular cross-sectional shape of the vessel at least substantially. The stent assembly also includes a patency zone having at least one patency stent structure located distal of the inlet stent structure, wherein the patency stent structure is self-expanding and has a patency radial force greater than the inlet radial force, and wherein the patency stent structure is coupled to the inlet stent structure such that the inlet stent structure is configured to be positioned upstream from a narrowing along the blood vessel and the patency stent structure is configured to be positioned at the narrowing of the blood vessel.

### CROSS-REFERENCE TO RELATED APPLICATIONS

This application claims the benefit of, and priority to, U.S. Provisional Patent Application Ser. No. 63/445,771 filed on Feb. 15, 2023. The entire contents of the foregoing application is incorporated by reference herein.

### TECHNICAL FIELD

The present disclosure relates to devices and methods for treating disorders associated with narrowing of a blood vessel lumen. The devices and methods can be directed to positioning a stent assembly in a dural venous sinus to restore and/or maintain a desired patency of the dural venous sinus.

### BACKGROUND

Stenoses of the intracranial dural venous sinuses are recently understood anatomical conditions that are associated with several diseases. For example, stenoses of the intracranial dural venous sinuses can be responsible for idiopathic intracranial hypertension (IIH), pulsatile tinnitus (PT), chronic migraines, headaches, cognitive decline, and in some cases blindness. Such stenoses have conventionally been managed relatively unsuccessfully with weight loss, high doses of carbonic anhydrase inhibitors, cerebral spinal fluid (CSF) shunting procedures, and optic nerve sheath fenestration (ONSF) surgery. In addition to having marginal long-term efficacy, these treatments also have a variety of associated risks and challenges.

Recently, conventional stents designed to treat lesions or other occlusions in blood vessels (e.g., arterial stents) have been implanted in the dural venous sinuses to treat stenoses. Such conventional stents are designed to reopen and maintain the patency of blood vessels with plaque, intimal hyperplasia, calcification, thrombus or the like that compromise blood flow. Such conventional stents are designed with sufficient radial forces and coverage to expand against these tough lesions to reopen the vessel and restore patency, as well as to protect against restenosis. Conventional stents are also designed to avoid compression of the stented portion of the vessel to avoid occluding the vessel. Moreover, conventional stents also have circular cross-sectional shapes, and many conventional stents are generally relatively short.

In practice, conventional stents are not well-suited for treating dural venous stenoses. For example, the region of the dural venous sinus immediately upstream from an implanted conventional stent collapses in 12%-25% of cases, which requires additional upstream stents to be implanted to reopen the collapse portion (known as “revision”). Conventional stents and their delivery to the target location also cause intracranial hemorrhaging in a material percentage of cases. Moreover, dural venous sinus stenoses often require several conventional stents to cover the narrowed portion and adjacent portions of the vessel. This requires highly skilled practitioners who can accurately operate a complex system of nested catheters to reach the target location and deploy multiple stents. As a result, conventional stents have several shortcomings for treating stenoses of the dural cerebral sinuses.

### BRIEF DESCRIPTION OF THE DRAWINGS

The accompanying figures, which are not necessarily to scale, form part of the specification and disclose various principles and advantages of systems, apparatuses, and methods of the present technology. Like reference numbers can refer to identical or similar features that have similar structure and functionality through the separate views. Advantages of embodiments of the systems, apparatuses, and methods will be apparent from the following detailed description. The drawings and the associated descriptions are provided to illustrate some but not all embodiments or examples of the present technology and do not limit the scope of the claimed inventions in any way.

FIG. 1A is a schematic illustration of a portion of the venous sinus and jugular vein vessels with an embodiment of a stent located within a portion of a delivery catheter.

FIG. 1B is a cross-sectional view of a normal unconstricted portion of a transverse sinus.

FIG. 1C is a cross-sectional view of a narrowed portion of a transverse sinus along line 1C-1C of FIG. 1A.

FIG. 1D is a cross-sectional view of a narrowed portion of a transverse sinus along line 1D-1D of FIG. 1A with an arachnoid granulation.

FIGS. 2A-2G are side views schematically illustrating some embodiments of stent assemblies in accordance with the present technology. FIGS. 2A-1, 2A-2, 2B-1, 2E-1 and 2G-1 schematically illustrate cross-sectional views of some embodiments of zones along stent assemblies of FIGS. 2A, 2B, 2E, 2F, 2G, 2H, and 2I respectively, in accordance with the present technology.

FIG. 3 is a side view schematically illustrating some embodiments of a stent assembly with a transition zone in accordance with the present technology, and FIG. 3-1 schematically illustrates cross-sectional views of some embodiments of zones along stent assemblies of FIG. 3 in accordance with the present technology.

FIG. 4 is a side view schematically illustrating some embodiments of a stent assembly with a transient pressure zone in accordance with the present technology, and FIG. 4-1 schematically illustrates cross-sectional views of some embodiments of zones along stent assemblies of FIG. 4 in accordance with the present technology.

FIG. 5A is a cross-sectional view schematically illustrating some embodiments of a zone of a stent assembly with flex regions in accordance with the present technology.

FIG. 5B is a cross-sectional view schematically illustrating some embodiments of a zone of a stent assembly with flex regions in accordance with the present technology.

FIG. 6 is a side view schematically illustrating some embodiments of stent assemblies in accordance with the present technology.

FIGS. 7A-7C are side views schematically illustrating some embodiments of stent assemblies in accordance with the present technology.

FIGS. 8A and 8B are side views schematically illustrating some embodiments of stent assemblies in accordance with the present technology.

FIGS. 9A-9D are side views schematically illustrating some embodiments of stent assemblies in accordance with the present technology.

FIG. 10 is a side view schematically illustrating some embodiments of a stent assembly in accordance with the present technology.

FIGS. 11A-11C schematically illustrate different cross-sectional shapes for a portion of a selected stent structures of some embodiments of a stent assembly in accordance with the present technology.

### DETAILED DESCRIPTION

### Overview

Systems, apparatuses, and methods of the present technology are related to treating disorders associated with narrowing of a blood vessel lumen, such as a compromised blood flow caused by a collapsed/contracted vessel wall and/or an occlusion. Some embodiments are directed to positioning a stent assembly in a dural venous sinus to maintain a desired patency of the dural venous sinus. However, the disclosed embodiments are merely examples of various embodiments of the present technology, and thus the disclosed embodiments may be used in other types of blood vessels, such as cardiovascular, pulmonary vascular, and/or peripheral vascular blood vessels. Therefore, specific structural and functional details disclosed herein are not to be interpreted as limiting, but merely as a basis for the claims and as a representative basis for teaching one skilled in the art to make and use the systems, apparatuses, and methods in appropriately detailed structure. Further, the terms and phrases used herein are not intended to be limiting, but rather they provide an understandable description of the systems, apparatuses, and methods.

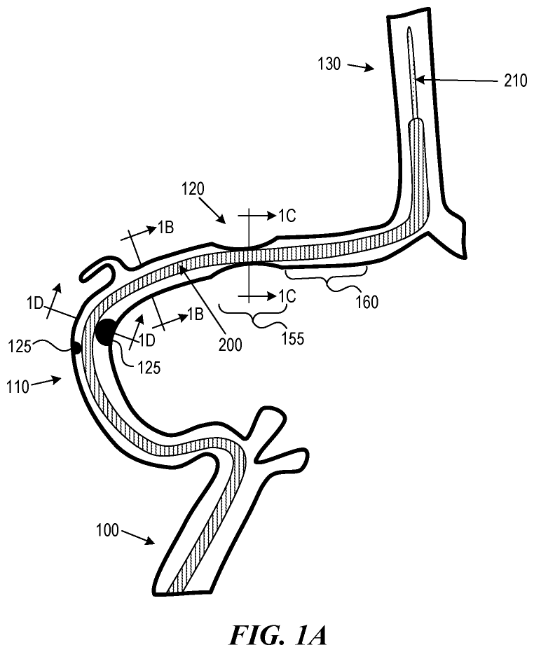

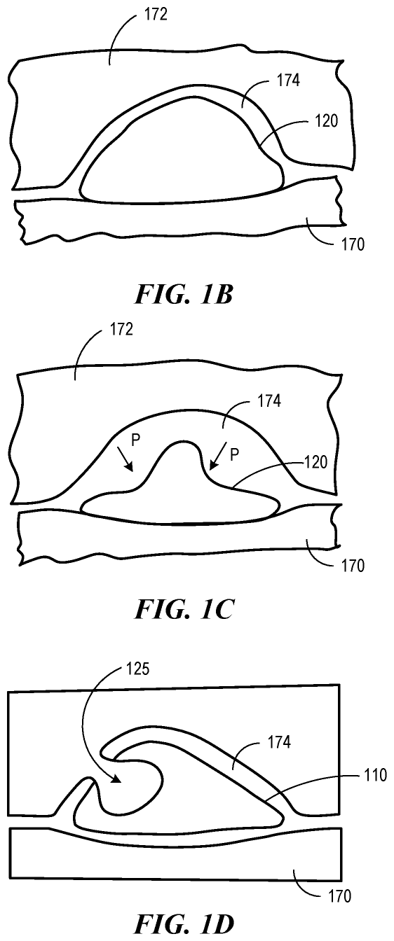

FIG. 1A illustrates a portion of the dural venous sinus system including the internal jugular vein 100, the sigmoid sinus 110, the transverse sinus 120, and the superior sagittal sinus 130. Blood flows from the superior sagittal sinus 130 toward the internal jugular vein 100. Dural venous stenoses often occur when the transverse sinus 120 has a narrowed portion 150 where the vessel wall is constricted compromising the vessel lumen and blood flow, which often results in an upstream location 160 between the narrowed portion 150 and the superior sagittal sinus 130. In addition, arachnoid granulations 125, tufts of arachnoid membrane invaginated into the dural sinuses through which cerebrospinal fluid (CSF) enters the venous system, can form throughout the dural venous sinuses causing an intrinsic narrowing or reduction in vessel cross sectional area. This can compromise blood flow through the dural venous sinuses and increase pressure within the vessel(s).

FIG. 1B is a cross-sectional view of a normal portion of a transverse sinus 120 without a narrowed portion, and FIG. 1C is a cross-sectional view of the narrowed portion 150 of the transverse sinus 120 along line 1C-1C of FIG. 1A. Referring to FIG. 1B, a normal transverse sinus 120 has a three-sided cross-sectional shape between bone 170 and the brain 172 with cerebral spinal fluid 174 in the space between the transverse sinus 120 and the brain 172. As used herein, such three-sided shapes are described as generally triangular as the sides may be curved inwardly and/or outwardly. Referring to FIG. 1C, dural venous stenoses often occur when the cerebral spinal fluid pressure increases (arrows P) to the extent that it overcomes the blood pressure in the transverse sinus and the structural integrity of the vessel wall. FIG. 1D depicts a narrowed portion 150 due to an arachnoid granulation in the sigmoid sinus 110. Some embodiments of the present technology are accordingly directed to deploying a stent assembly via a delivery catheter 200 over a guide wire 210 along the sigmoid sinus 110, the transverse sinus 120 and optionally into the superior sagittal sinus 130 to restore patency to the narrowed portion 150 of the dural venous sinuses.

The present technology addresses the shortcomings of implanting conventional stents in a dural venous sinus. Unlike plaque, thrombus, intimal hyperplasia, and calcification that block or clog the vessel, dural venous sinus stenoses predominantly occur when an increase in extravascular pressure of the cerebral spinal fluid exceeds the intravascular pressure and the inherent crush resistive force of the sinus wall. As such, instead of requiring a radial force sufficient to push plaque, calcifications or other occlusions radially outward to restore patency, the radial forces necessary to open a dural venous sinus only need to exceed these extravascular forces. In some cases, dural venous sinus stenoses may also be caused by arachnoid granulations within the sinus, but again these lesions require only low radial forces to restore patency. In addition, the transverse sinus 120 and superior sagittal sinus 130 have generally triangular cross-sectional shapes, whereas conventional arterial and venous stents generally expand to have circular cross-sectional shapes when deployed in a vessel. As a result, the present inventors are the first to recognize that these differences between stenoses of the dural venous sinuses and other types of stenoses in other vessels are some of the reasons why conventional stents have several shortcomings in treating stenoses of the dural venous sinuses.

For example, the region of the dural venous sinus immediately upstream from an implanted convention stent collapses in 12%-25% of cases, as explained above. The present inventors believe that the large radial forces exerted by conventional stents on the transverse sinus vessel wall and the circular cross-sectional shape of conventional stents causes a low-pressure region at the upstream end of the stent, which in turn causes the portion of the transverse sinus wall to collapse and restrict flow immediately upstream of the stent. Further, the circular cross-sectional shape and the large radial forces of conventional stents are also likely contributors to intracranial hemorrhaging and other undesirable effects, such as high forces on the vessel walls (dura mater, periosteum) that causes pain or distortion of the vessel walls. Conventional stents, moreover, generally do not have the necessary length or the appropriate cross-sectional area to stent the entire target area, which may be approximately 12 cm or more if stenting from the superior sagittal sinus to the sigmoid sinus (e.g., 12 cm to 20 cm, or 14 cm to 18 cm. As a result, several conventional stents are generally required to cover the narrowed portion of the vessel, which requires highly skilled practitioners to operate a complex system of nested catheters to accurately deploy multiple stents.

Another aspect of the transverse sinus 120 is that under normal physiologic conditions a portion of the vessel reacts almost immediately to increases in cerebral spinal fluid pressure (coughing, Valsalva, snoring, etc.) by developing a focal stenosis in the lateral transverse sinuses allowing for temporary pressurization of the dural venous sinuses proximally that resolves when the cerebral spinal fluid pressure resolves. The temporary development of focal stenoses in both lateral transverse sinuses allows pressure to build within the upstream dural venous sinuses and thus enables the remainder of the system (that is now pressurized) to resist collapse due to cerebral spinal fluid pressure spikes. The large radial forces of conventional stents do not compress in response to such changes in cerebral spinal fluid pressure, which limits how much pressure can develop in the proximal venous sinuses and therefor their ability to resist collapse during cerebrospinal fluid pressure spikes is hindered.

The present technology is directed to stent assemblies and method of treating narrowed portions of blood vessels that addresses these shortcomings of conventional stents. In some embodiments, a stent assembly for implantation into a blood vessel includes at least an inlet zone and a patency zone. The inlet zone has at least one inlet stent structure, which is self-expanding and has an inlet radial force configured to conform to a non-circular cross-sectional shape of the vessel at least substantially. The patency zone has at least one patency stent structure located distal of the inlet stent structure. The patency stent structure is self-expanding and has a patency radial force greater than the inlet radial force, and the patency stent structure is coupled to the inlet stent structure such that the inlet stent structure is configured to be positioned upstream from a narrowing along the blood vessel and the patency stent structure is configured to be positioned at the narrowing of the blood vessel.

In some embodiments, a stent assembly for implantation into a blood vessel includes at least an inlet zone and a patency zone. The inlet zone has at least one inlet stent structure, which is self-expanding and is configured to at least substantially conform to a non-circular cross-sectional shape of the vessel. The patency zone has at least one patency stent structure located distal of the inlet stent structure. The patency stent structure is self-expanding and is configured to expand to an expanded shape different than the non-circular cross-sectional shape of the vessel whereby the vessel at least substantially conforms to the patency stent structure. The patency stent structure is coupled to the inlet stent structure such that the inlet stent structure is configured to be positioned upstream from a narrowing along the blood vessel and the patency stent structure is configured to be positioned at the narrowing of the blood vessel.

In some embodiments, a stent assembly for implantation into a blood vessel includes an inlet zone and a patency zone. The inlet zone has at least one inlet stent structure, wherein the inlet stent structure is self-expanding and has an inlet radial force that compresses with increasing external pressure on the blood vessel. The patency zone has at least one patency stent structure that is self-expanding and has a patency radial force greater than the inlet radial force. The patency radial force is sufficient to maintain patency of the blood vessel when external pressure on the blood vessel increases.

The present technology is also directed to methods of treating an indication caused by a narrowing along a dural venous sinus. Some embodiments of methods in accordance with the present technology include positioning a stent assembly in a dural venous sinus such that (a) an inlet zone of the stent assembly is at an upstream location from a narrowed portion of the dural venous sinus relative to blood flow through the dural venous sinus and (b) a patency zone of the stent assembly is at the narrowed portion of the dural venous sinus. The method further includes expanding an inlet stent structure of the inlet zone and expanding a patency structure of the patency zone in the dural venous sinus. The inlet stent structure is expanded at the upstream location to have a triangular cross-section shape that at least substantially approximates a generally triangular cross-sectional shape of the dural venous sinus at the upstream location, and the inlet stent structure has an inlet radial force. The patency stent structure is expanded at the narrowed portion of the dural venous sinus such that the patency stent structure increases a cross-sectional area of the narrowed portion, and the patency stent structure has a patency radial force greater than the inlet radial force.

In some embodiments, methods in accordance with the present technology include positioning a stent assembly in a dural venous sinus such that (a) an inlet zone of the stent assembly is at an upstream location from a narrowed portion of the dural venous sinus relative to blood flow through the dural venous sinus and (b) a patency zone of the stent assembly is at the narrowed portion of the dural venous sinus. The method further includes (a) expanding an inlet stent structure of the inlet zone such that the inlet stent structure at least substantially conforms to a non-circular cross-sectional shape of the upstream location of the dural venous sinus and (b) expanding a patency structure of the patency zone to an expanded shape such that a non-circular cross-sectional shape of the narrowed portion of the dural venous sinus at least substantially conforms to the expanded shape of the patency stent structure. The patency stent structure is expanded at the narrowed portion of the dural venous sinus such that the patency stent structure increases a cross-sectional area of the narrowed portion.

In some embodiments, the inlet stent structure has at least a generally circular cross-sectional shape in an unconstrained expanded state and is configured to flex into a generally triangular shape as it expands in contact with the blood vessel, and the patency stent structure has at least a generally circular cross-sectional shape in an unconstrained expanded state and is configured to flex into a generally triangular shape as it expands in contact with the blood vessel. In some embodiments, the inlet structure has at least a generally triangular cross-sectional shape in an unconstrained expanded state and is configured to at least substantially approximate a triangular shape of the upstream location, and the patency stent structure has at least a generally circular cross-sectional shape in an unconstrained expanded state and is configured to expand into a generally circular cross-section shape as it expands in contact with the blood vessel. In some embodiments, the inlet stent structure has at least a generally circular cross-section shape in an unconstrained expanded state and is configured to flex into a generally triangular cross-sectional shape as it expands in contact with a blood vessel.

In some embodiments, the inlet radial force of the inlet stent structure is approximately 0.0005 N/mm to approximately 2 N/mm, and the patency radial force of the patency stent structure is approximately 0.001 N/mm to approximately 3 N/mm. In some embodiments, the patency radial force of the patency stent structure is configured to limit constriction of the blood vessel when external pressure on the blood vessel increases.

In some embodiments, the stent assembly further comprises an outlet zone including at least one outlet stent structure located distal of the patency stent structure, wherein the outlet stent structure is self-expanding and has an outlet radial force less than the patency radial force. In some embodiments, the stent assembly further comprises a transition zone between the inlet zone and the patency zone, wherein the transition zone has a transition stent structure having a transition radial force greater than the inlet radial force of the inlet stent structure and less than the patency radial force of the patency stent structure. In some embodiments, the stent assembly further comprises a transient pressure zone adjacent to the patency zone having a transient stent structure with a transient radial force configured to constrict during structure physiologic spikes in cerebral spinal fluid pressure.

In some embodiments, the inlet structure comprises an inlet ring having struts and the patency stent structure comprises a patency ring having struts. In some embodiments, the inlet ring is a cut or etched hypo-tube having inlet struts and the patency stent structure is cut or etched hypo-tube having patency struts that have a larger cross-sectional area than the inlet struts. In some embodiments, the inlet structure comprises a braided mesh and the patency stent structure comprises a patency ring having struts. In some embodiments, the inlet stent structure comprises an inlet braided mesh section having the inlet radial force and the patency stent structure comprises a patency braided mesh section having the patency radial force. The inlet braided mesh portion can be a different braided mesh than the patency braided mesh portion, or the inlet braided mesh portion and the patency braided mesh portion can be part of a single, integrated braided mesh in which the braid angle, wire count, wire thickness and/or other parameters are different between the inlet braided mesh portion and the patency braided portion.

In some embodiments, a stent assembly for implantation into a blood vessel includes at least an inlet zone and a patency zone. The inlet zone has at least one inlet stent structure, which is self-expanding and has an inlet radial force configured to conform to a non-circular cross-sectional shape of the vessel at least substantially. The patency zone has at least one patency stent structure located distal of the inlet stent structure (e.g., downstream from the inlet stent structure with respect to the direction of blood flow). The patency stent structure is self-expanding and has a patency radial force greater than the inlet radial force, and the patency stent structure is coupled to the inlet stent structure such that the inlet stent structure is configured to be positioned upstream from a narrowing along the blood vessel and the patency stent structure is configured to be positioned at the narrowing of the blood vessel. The stent assembly can further comprise one or more additional patency zones and/or one or more outlet zone(s), transition zone(s), intermediate transition zone(s) and/or transient pressure zone(s). The outlet zone can include at least one outlet stent structure located distal of the patency stent structure, wherein the outlet stent structure is self-expanding and has an outlet radial force less than the patency radial force. A transition zone can be between the inlet zone and the patency zone, wherein the transition zone has a transition stent structure having a transition radial force greater than the inlet radial force of the inlet stent structure and less than the patency radial force of the patency stent structure. An intermediate transition zone can be located within a patency zone or between patency zones. The transient pressure zone can be within a patency zone, between a patency zone and the outlet zone, or between a patency zone and any transition zone, intermediate transition zone, or inlet zone. The transient pressure zone can have a transient stent structure with a transient radial force configured to constrict during physiologic spikes in cerebral spinal fluid pressure. In some embodiments, the transition stent structure can be coupled to a distal portion of the inlet stent structure and a proximal portion of the patency stent structure, and the transient stent structure can be coupled to a distal portion of the patency stent structure and a proximal portion of the outlet stent structure.

In some embodiments, the patency zone has a similar radial force to the inlet zone, but the inlet zone is constructed such that it is more conformal to the shape of the vessel than the patency zone. This can be accomplished by having an inlet zone with more struts radially around the stent structure and/or having longitudinally longer stent structures.

Several aspects of the present technology are also directed to methods of treating an indication caused by a narrowing along a dural venous sinus. In some embodiments, a method comprises positioning a stent assembly in a dural venous sinus such that (a) an inlet zone of the stent assembly is at an upstream location from a narrowed portion of the dural venous sinus relative to blood flow through the dural venous sinus and (b) a patency zone of the stent assembly is at the narrowed portion of the dural venous sinus. The method can include expanding an inlet stent structure of the inlet zone to have a three-sided cross-sectional shape (e.g., at least generally triangular) that at least substantially approximates a generally triangular cross-sectional shape of the dural venous sinus at the upstream location, wherein the inlet stent structure has an inlet radial force. The method can further include expanding a patency stent structure of the patency zone at the narrowed portion of the dural venous sinus such that the patency stent structure increases a cross-sectional area of the narrowed portion, wherein the patency stent structure has a patency radial force greater than the inlet radial force.

In some embodiments of the method, the inlet stent structure has a circular cross-sectional shape in an expanded unconstrained state and expanding the inlet stent structure comprises self-expanding the inlet stent structure such that it at least substantially approximates the generally triangular cross-section shape of the upstream location.

In some embodiments of the method, the inlet stent structure is coupled to the patency stent structure before positioning the stent assembly in the dural venous sinus such that a single stent assembly is positioned along the upstream location and the narrowed portion of the dural venous sinus.

In some embodiments the indication is papilledema. In some embodiments, the indication is pulsatile tinnitus. In some embodiments, the indication is headache or chronic migraine.

In some embodiments of the method, the inlet stent structure has a circular cross-sectional shape in an expanded unconstrained state and expanding the inlet stent structure comprises self-expanding the inlet stent structure such that it at least substantially approximates the generally triangular cross-sectional shape of the upstream location, the patency stent structure has a circular cross-sectional shape in an expanded unconstrained state and expanding the patency stent structure comprises self-expanding the patency stent structure such that it at least substantially approximates a generally triangular cross-sectional shape of the narrowed portion of the dural venous sinus. In some embodiments of the method, upon expansion the generally triangular cross-sectional shape of the inlet stent structure is different than the generally triangular cross-sectional shape of the patency stent structure.

In some embodiments of the method, the inlet stent structure has a circular cross-sectional shape in an expanded unconstrained state and expanding the inlet stent structure comprises self-expanding the inlet stent structure such that it at least substantially approximates the generally triangular cross-sectional shape of the upstream location, the patency stent structure has a circular cross-sectional shape in an expanded unconstrained state and expanding the patency stent structure comprises self-expanding the patency stent structure such that it expands into a generally circular cross-section shape as it expands in contact with the blood vessel.

In some embodiments of the method, positioning the stent assembly in the dural sinus further comprises locating an outlet zone of the stent assembly at a downstream location from the narrowed portion of the dural venous sinus relative to blood flow through the dural venous sinus, and wherein the outlet zone has an outlet stent structure with an outlet radial expansion force less than the patency expansion force. In some embodiments of the method, the outlet stent structure of the outlet zone is expanded at the downstream location such that the outlet stent structure has a cross-sectional shape approximating a cross-sectional shape of the downstream location.

Although some embodiments are directed to being positioned in a dural venous sinus, aspects of the present technology can be applied using one or more stents for implantation into other vessels of the body of a human or animal subject. Several embodiments, as noted above, are directed to implanting one or more stents that restore blood flow within the venous sinuses, and in particular the transverse sinus 120, the sigmoid sinus 110 and/or the superior sagittal sinus 130.

Still further in accordance with the present technology, the one or more stent structures may include an inlet zone. The inlet zone is constructed to enable a smooth transition from the native vessel into the stent and stented portion of the vessel. The inlet zone can at least in part, conform to the general shape of the native vessel. The inlet zone can conform to the native vessel without substantially over dilating the vessel to reduce disturbances of the blood flow from the native vessel into the stented region.

Still further in accordance with the present technology, the one or more stent structures may include one or more transition zones. The transition zone(s) provide a transition from the inlet zone to the patency zone of the stent with increased radial force compared to the inlet zone. This also provides for reduced turbulence within the stent.

Still further in accordance with the present technology, the one or more stent structures may include one or more intermediate transition zones. The intermediate transition zone(s) provide a transition within a patency zone or between one or more patency zones, The intermediate transition zone can have a reduced radial force compared to one or more adjacent patency zones.

Still further in accordance with the present technology, the one or more stent structures include one or more patency zones (e.g., body zones) which supply the desired level of radial force to restore and maintain patency of the vessel within the narrowed or target area(s) and optionally adjacent or more distant regions of the vessel.

Still further in accordance with the present technology, the one or more stents may include one or more transient pressure zones. The transient pressure zone(s) flex in response to physiologic changes (e.g., reduce the cross-sectional area) when external pressure on the vessel wall increases urging the vessel cross-sectional area to be reduced, and then urge the vessel wall radially outward when the external pressure abates. This allows the vessel to have a natural response to transient pressure increases of the cerebral spinal fluid. An example is when there is a transient spike in cerebral spinal fluid pressure, at least a portion of the stented vessel could decrease in cross-sectional area and when the pressure abates, that portion of the stented vessel recovers (e.g., increases in cross-sectional area).

Still further in accordance with the present technology, the one or more stent assemblies may include an outlet zone or several outlet zones. The outlet zone(s) serve to reduce flow disturbances as the blood exits the stented region.

Still further in accordance with the present technology, the one or more stent assemblies may include flex regions to enable at least a portion of the stent to conform to the vessel wall more easily without delivering undesirable, undue, or excessive pressure to the vessel wall.

Still further in accordance with the present technology, the one or more stent assemblies may be constructed as round (e.g., circular) in cross-sectional profile and/or round (e.g., circular) when deployed in free space and when deployed in a vessel at least part of the stent conforms to the general shape of the native vessel.

Still further in accordance, the one or more stent assemblies may be constructed in the shape of the relative shape(s) of the vessel(s) into which the stent is to be deployed when unconstrained in free space and or flex to approximate the relative shape(s) of the vessel(s) into which the stent is to be deployed.

Still further in accordance with the present technology, the one or more stent assemblies may be constructed such that at least a portion of the stent can be deployed into the vessel and then, if desired, recaptured back into a delivery catheter.

Still further in accordance with the present technology, one or more stent assemblies may be constructed such that when it is partially constrained it has higher radial forces to resist further compression and facilitate adequate venous sinus decompression than when it is fully expanded to nominal.

Still further in accordance with the present technology, the one or more stent assemblies can be constructed of a shape memory material, such as NiTi or NiTi alloy to enable self-expansion in the vessel when deployed from a delivery catheter.

### DETAILED DESCRIPTION OF SELECTED EMBODIMENTS OF THE PRESENT TECHNOLOGY

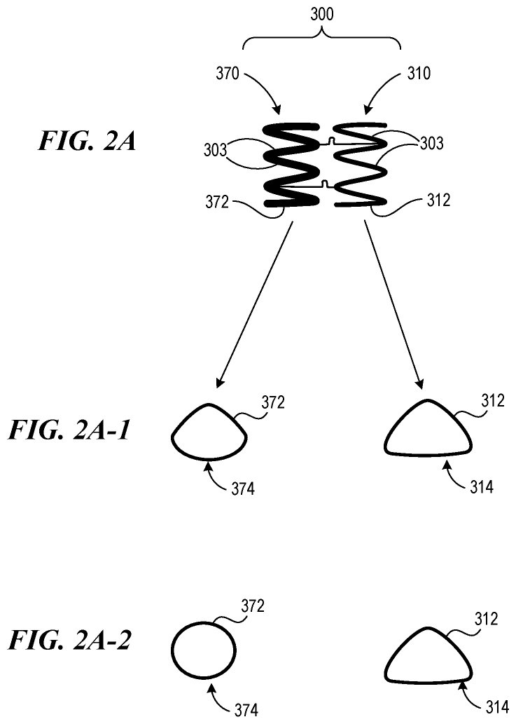

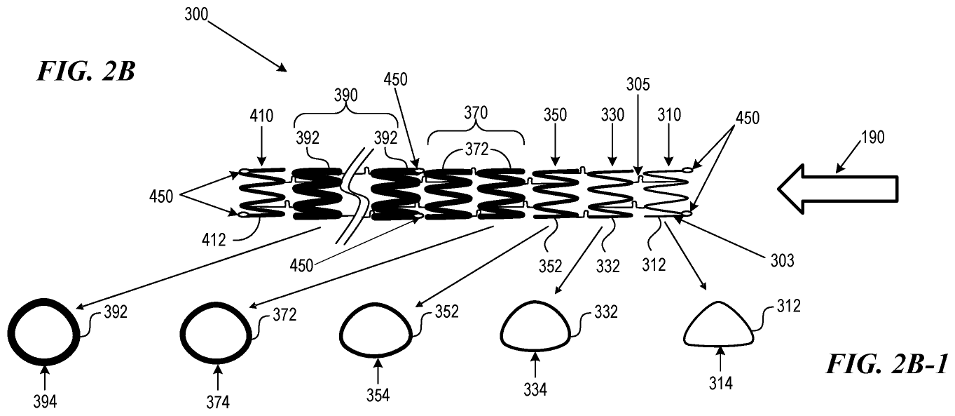

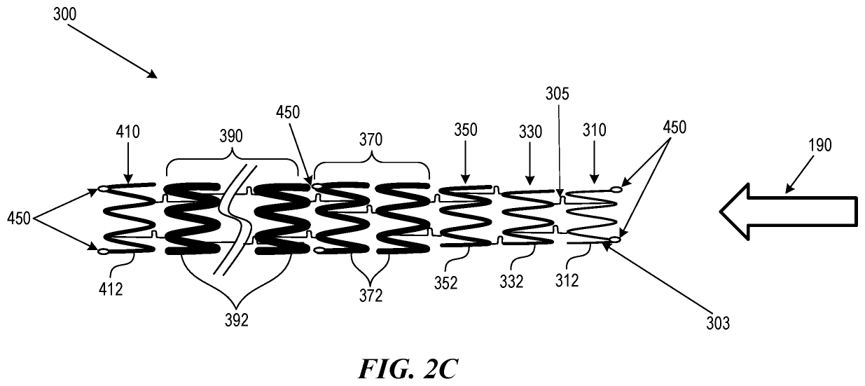

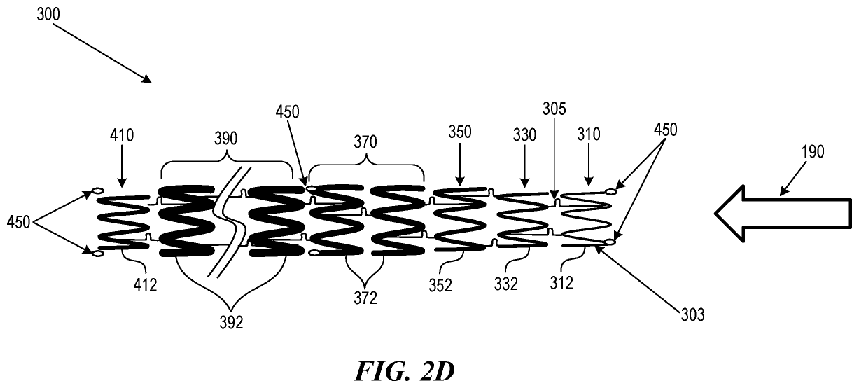

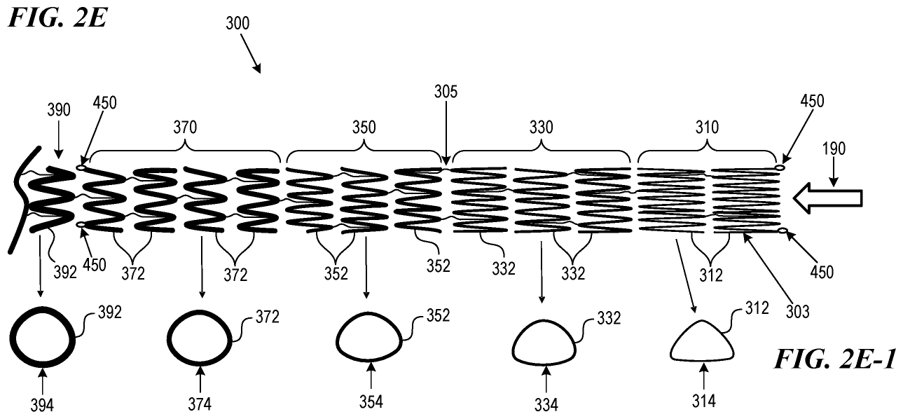

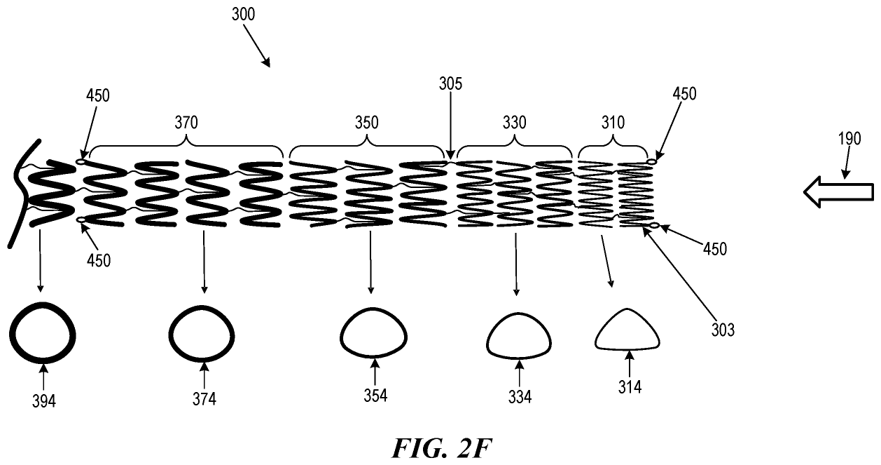

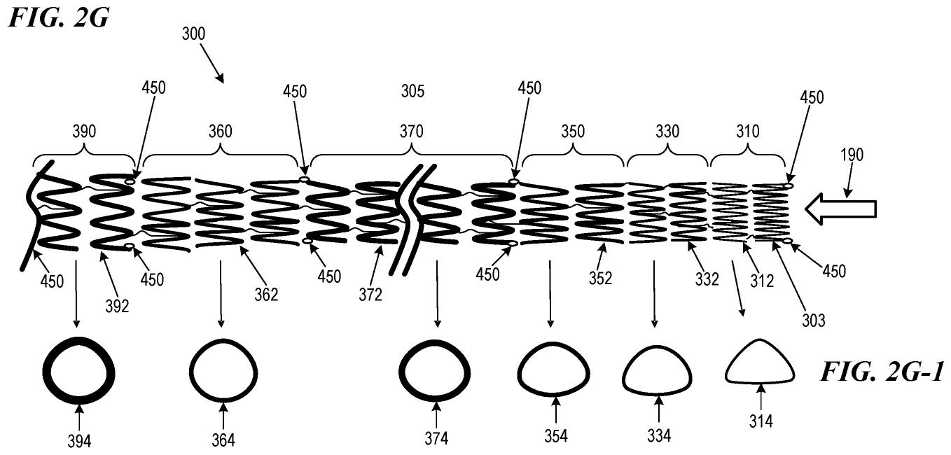

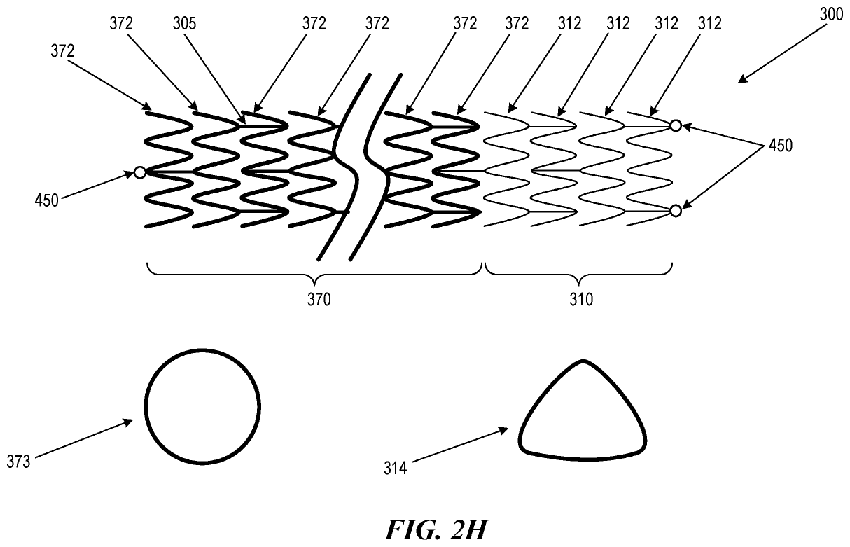

FIGS. 2A-2I are side views schematically illustrating some embodiments of stent assemblies 300 in accordance with the present technology. Referring to FIG. 2A, the stent assembly 300 can include one or more zones, and in some embodiments the stent assembly 300 includes at least two zones having different expansion properties. In some embodiments, the stent assembly 300 has an inlet zone 310 and a patency zone 370 (e.g., body zone). As shown in FIG. 2A, the inlet zone 310 includes at least one inlet stent structure 312 that is self-expanding and is configured to flex and thereby at least substantially approximate a non-circular cross-sectional shape of a portion of a vessel at the inlet zone 310. The inlet stent structure 312 can have an inlet radial force. The patency zone 370 includes at least one patency stent structure 372 that is self-expanding and is configured to expand to an expanded shape that increases the cross-sectional area of another portion of the vessel at the patency zone 370. For example, the portion of the vessel at the patency zone 370 can expand and at least substantially conform to the expanded shape of the patency stent structure 372. The patency stent structure 372 can have a patency radial force greater than the inlet radial force. The stent structures 312/372 can be rings comprising struts 303 and/or braided meshes. For example, the rings can be cut or etched from hypo-tubes and the braided meshes can be wire or polymeric braids.

In some embodiments, the patency stent structure 372 is directly coupled to the inlet stent structure 312 by flexible links 305 such that the inlet stent structure 312 is positioned upstream from a narrowed portion of a blood vessel and the patency stent structure is positioned at the narrowed portion of the blood vessel. In some embodiments, such as several described below, the patency stent structure 372 is indirectly coupled to the inlet stent structure 312 with other zones having other stent structures between the patency stent structure 372 and the inlet stent structure 312 (see, e.g., FIG. 2B). In either case, the inlet stent structure 312 and the patency stent structure 372 are “coupled” to each other. In some embodiments, the patency stent structure 372 is directly connected to the inlet stent structure 312 without the links 305 via welds, adhesives, fasteners (e.g., sutures, fibers), or other suitable techniques.

FIG. 2A-1 is a cross-sectional view of some embodiments of the inlet stent structure 312 and the patency stent structure 372 upon deployment. In some embodiments, the inlet stent structure 312 has at least a generally circular cross-sectional shape in an unconstrained expanded state (not shown) and is configured to flex into a generally triangular shape 314 (shown) as it expands in contact with the upstream location of the blood vessel, and the patency stent structure 372 has at least a generally circular cross-sectional shape in an unconstrained expanded state (not shown) and is configured to flex into a different triangular shape 374 as it expands in contact with the narrowed portion of the blood vessel. In some embodiments, the inlet stent structure 312 has at least a generally triangular cross-sectional shape in an unconstrained expanded state and is configured to have a generally triangular shape 314 at least substantially approximating a generally triangular shape of the upstream location. In some embodiments as shown in FIG. 2A-2, the patency stent structure 372 has at least a generally circular cross-sectional shape in an unconstrained expanded state (not show) and is configured to expand into a generally circular cross-section shape (not shown) as it expands in contact with narrowed portion the blood vessel. As shown in FIG. 2A-2, upon expansion the inlet stent structure 312 has a generally triangular cross-sectional shape 314 and the patency stent structure 372 has at least a generally circular cross-sectional shape 374. The inlet stent structure 312 can accordingly flex and adapt to a generally triangular cross-sectional shape of the portion of the vessel at the inlet zone 310, while the patency stent structure 372 at least substantially maintains a generally circular cross-sectional shape such that a portion of the vessel having a non-circular cross-sectional shape at the patency zone 370 at least substantially conforms to the generally circular cross-sectional shape of the expanded patency stent structure 372.

The inlet radial force of the inlet stent structure 312 is selected such that the inlet stent zone 310 conforms to the natural shape of the upstream location at least substantially. When the stent assembly 300 is implanted in the dural venous sinus such that the inlet zone 310 is in the transverse sinus 120, the inlet zone 310 is expected to have a generally triangular shape that at least substantially approximates the triangular cross-sectional shape of the corresponding portion of the transverse sinus 120. This feature is expected to reduce low-pressure regions at the inlet of the stent assembly 300 because the inlet zone 310 does not substantially over expand and deform relative to the upstream blood vessel. As a result, the stent assembly 300 is expected to reduce or prevent the blood vessel from collapsing upstream from the stent assembly, which should reduce the rate of “revision” procedures.

The patency radial force of the patency stent structure 372 is selected such that the patency zone 370 restores the patency of the narrowed portion of the blood vessel. The patency radial force, for example, can be selected to be greater than the pressure of the cerebral spinal fluid to expand the narrowed portion of the vessel outward and restore patency. As a result, the cross-sectional shape of the expanded patency stent structure 372 reshapes the narrowed portion of the blood vessel to restore blood flow therethrough.

In some embodiments, the inlet radial force of the inlet stent structure 312 is approximately 0.0005 N/mm to approximately 2 N/mm, and the patency radial force of the patency stent structure 372 is approximately 0.001 N/mm to approximately 3 N/mm. In some embodiments, the patency radial force of the patency stent structure 372 is configured to limit constriction of the blood vessel when external pressure on the blood vessel increases.

FIG. 2B is a side cross-sectional view of some embodiments of a stent assembly 300. In this embodiment, the stent assembly includes the inlet zone 310, an optional first transition zone 330, an optional second transition zone 350, a first patency zone 370 (e.g., first body zone), a second patency zone 390 (e.g., second body zone), and an outlet zone 410. Each zone can have one or more stent structures. For example, the inlet zone 310 can include an inlet stent structure 312, the first transition zone 330 can include a first transition stent structure 332, the second transition zone 350 can include a second transition stent structure 352, the first patency zone 370 can include a first patency stent structures 372, the second patency zone 390 can include a second patency stent structure 392, and the outlet zone 410 can include an outlet stent structure 412.

The number of zones and the number of stent structures in each zone can vary depending upon the application. Each stent structure can include at least one ring comprising struts 303 and/or at least one braided wire mesh. In some embodiments, the stent structures of at least some zones of the stent assembly 300 may have a round cross-sectional shape when unconstrained in free space (i.e., an unconstrained fully expanded state), and when deployed in the vasculature at least some of the stent structures of the stent assembly 300 may flex to at least generally approximate the relative shape(s) of the corresponding portions of the native vessel(s). In some embodiments, some stent structures in one or more of the zones may have an unconstrained expanded cross-sectional shape that is different than the cross-sectional shape of a corresponding portion of the vessel at the target site, and upon expansion the corresponding portion of the vessel at least substantially conforms to the unconstrained cross-sectional shape of such stent structures. In some embodiments, the stent structures of at least some zones of the stent assembly 300 may have the relative shape(s) of the corresponding portion(s) of the blood vessel(s) in an unconstrained fully expanded state and when deployed in the vasculature. In some embodiments, the stent structures of at least some zones of the stent assembly 300 may have the relative shape(s) of the corresponding portion(s) of the blood vessel(s) in an unconstrained fully expanded state and when deployed in the vasculature flex to further approximate the relative shape(s) of the corresponding portion(s) of the vessel(s). The stent assembly 300 can also include interconnecting links 305 connecting adjacent stent structures together. Alternatively, adjacent stent structures may be directly connected to each other without the use of links 305. The number of links 305 connecting stent structures may vary along the length of the stent assembly 300. Links 305 may be arranged linearly along the longitudinal length of the stent assembly 300, in a helical pattern, or any combination.

The stent structures in the different zones can have different cross-sectional profiles and physical properties along the length of the stent assembly 300. For example, in the embodiment shown in FIGS. 2B and 2B-1, the stent structures 312, 332, 352, 372, 392 can progress from being a relatively lower radial force stent structure (e.g., the inlet stent structure 312) to a being a relatively higher radial force stent structure (e.g., the second patency stent structure 392). As a result, in some embodiments the cross-sectional shapes of the stent structures upon deployment can be different along the length of the stent assembly according to the radial forces of the stent structures and the cross-sectional shapes of the corresponding areas of the vessel(s) where the stent assembly 300 is deployed. Referring to FIG. 2B-1, upon deployment in the sigmoid sinus 110 (FIG. 1) the second patency stent structure 392 can have a nearly or generally circular cross-sectional shape 394 or a rounded-quadrangle cross-sectional shape 414 that at least substantially approximates the shape of the sigmoid sinus 110. Whereas upon deployment in the transverse sinus 120 (FIG. 1) the inlet stent structure 312 can have a generally triangular shape 314 that at least substantially approximates the triangular shape of the transverse sinus 120.

Several embodiments of the stent assembly 300 manage the blood flow into and through the stent assembly 300 to reduce (e.g., minimize or eliminate) flow disturbances or turbulence. To achieve this result, the different zones of the stent assembly 300 can be generally shaped like the corresponding areas of the blood vessel(s) where the stent assembly 300 is to be implanted and/or the different zones of the stent assembly 300 can flex upon expansion to at least substantially approximate the native cross-sectional shapes of corresponding portions of healthy native vessel(s). The general cross-sectional shape may extend throughout the length of the stent assembly 300 or only for a specific length of the stent assembly 300 such that flow disturbances are mitigated or eliminated. The reduction/elimination of flow disturbances is expected to reduce “revisions”—typically the collapse of the native vessel adjacent the inflow side of the stent assembly 300. For example, in some embodiments the inlet zone 310, the optional first and second transition zones 330/350, and the outlet zone 410 are configured to flex to at least substantially approximate the corresponding areas where these zones are to be positioned along the sigmoid sinus 110 and the transverse sinus 120 and/or in the superior sagittal sinus 130. In contrast, the first and second patency zones 370/390 are configured to reshape the narrowed portions of the blood vessel(s) to have at least substantially the shapes of the first and second patency zones 370/390 in their unconstrained expanded states to restore blood flow through the vessels.

In some embodiments, the inlet zone 310 can be a shape memory material formed from a cut or etched hypo-tube or braided filaments to have either a generally triangular cross-sectional shape or a generally circular cross-sectional shape in an unconstrained expanded state and when expanded within the vessel(s) is sufficiently elastic to conform to the cross-sectional shape of the vessel such that the shape of the corresponding portion of the native vessel is at least substantially unaltered (e.g., at least substantially approximates the cross-sectional shape of the portion of the vessel where the inlet zone 310 is implanted). The inlet zone 310 can have outward radial forces from approximately 0.0005 N/mm to approximately 2 N/mm, or from approximately 0.05 N/mm to approximately 0.5 N/mm. The inlet zone 310 may be comprised of one or more stent structures and extend for a length of approximately 1 mm to approximately 100 mm, or from approximately 5 mm to approximately 25 mm. The inlet zone 310 may be coupled directly to the patency zone 370, or the inlet zone 310 and the patency zone 370 can be indirectly coupled to each other with one or more other zones having intermediate radial force stent structures and links 305 between inlet zone 310 and patency zone one 370. In some embodiments, the radial forces of the zones can sequentially increase from the inlet zone 310 through the second patency zone 390 along a continuous gradient and/or multiple steps.

Referring to FIG. 2B, the first transition zone 330 and second transition zone 350 can provide a gradual increase in radial force between that of inlet zone 310 and the first patency zone 370. For example, the first patency zone 370 can have a radial force of approximately 0.001 N/mm to approximately 3 N/mm, and at least a portion of the first transition zone 330 may have a radial force of approximately 0.001 N/mm to approximately 1.5 N/mm. Also, at least a portion of a second transition zone 350 may have a radial force of approximately 0.002 N/mm to approximately 2.5 N/mm. As such, at least a portion of the first transition zone 330 can have a higher radial force than the inlet zone 310 and lower radial force than at least a portion of the second transition zone 350. Similarly, at least a portion of the second transition zone 350 can have a lower radial force than at least a portion of the first patency zone 370. Referring to FIG. 2B-1, the deployed profiles within the vessel of the first transition zone 330 and the second transition zone 350 are shown as a first transition zone profile 334 and a second transition zone profile 354, respectively. The entire transition zone may extend for a length of approximately 2 mm to approximately 50 mm or more.

In some embodiments, one or more of the inlet zone 310, transition zones 330/350, patency zones 370/390, and the outlet zone 410 have variable radial force such that the stent assembly 300 will not collapse to less than 3 mm in diameter even in supraphysiological conditions. For example, if the radial force when fully expanded is between approximately 0.05 N/mm and approximately 1.5 N/mm, when the stent assembly 300 is compressed to 3 mm in diameter or less (or 9 mm perimeter) the radial force will be approximately 2 N/mm or more. This would allow for sufficient venous sinus decompression regardless of variations in the cerebral spinal fluid pressure without undue distortion in the native dural venous sinus at maximum expansion.

In certain embodiments, the patency zones 370/390 of the stent assembly 300 have sufficient radial force to hold the vessel open under the highest anticipated external pressure, as well as internal requirements, such as to maintain sufficient blood flow through the vessel. The patency zones 370/390 of the stent assembly 300 can have stent structures (e.g., sections) with a consistent radial force or stiffness. Additionally, one or more zones can have different radial forces (i.e., multiple patency zones), or one or more zones can have continuously changing radial forces and/or consistent or varying cross-sectional areas. In this manner, the entire length of the stent assembly 300 can effectively have a varying degree of radial forces and provide for lengths with consistent radial forces and/or with varying radial forces.

Referring to FIG. 2B, the radial forces exerted by the patency zones 370/390 is typically from approximately 0.001 N/mm to approximately 3 N/mm, or from approximately 0.04 N/mm to approximately 2 N/mm. For example, the first patency zone 370 may have the necessary radial force to restore patency along at least a portion of the superior sagittal sinus 130 and/or transverse sinus 120 (FIG. 1), and the second patency zone 390 may have a different radial force to restore patency along at least a portion of the transverse sinus 120 and/or sigmoid sinus 110. For example, the first patency radial force of the first patency zone 370 may be from approximately 0.001 N/mm to approximately 2.5 N/mm and the second radial force of the second patency zone 390 may be from approximately 0.005 N/mm to approximately 3 N/mm. The length of the patency zones, individually or combined, can be from approximately 50 mm to approximately 200 mm for placement from near or at the torcula, including placement within the superior sagittal sinus 130, and extending into the sigmoid sinus 110.

FIG. 2C is a side view schematically showing some embodiments of the stent assembly 300 in which the cross-sectional area of at least some of the stent structures increases from the inlet zone 310 toward the outlet zone 410. For example, the inlet stent structure 312 of the inlet zone 310 can have an inlet cross-sectional dimension (e.g., from a center point to a strut 303 or wire of the stent structure) and the second patency zone 390 can have a second patency cross-sectional dimension greater than the inlet cross-sectional dimension. The cross-sectional dimensions of the transitions zone 330/350 can be greater than that of the inlet zone 310 but less than that of the second patency zone 390, and the cross-sectional dimension of the first patency zone 370 can be less than that of the second patency zone 390. In some embodiments, the cross-sectional dimension of each zone can sequentially increase from the inlet zone 310 to the second patency zone 390. In some embodiments, each stent structure within each zone can have the same cross-sectional dimension, or in the case of zones with multiple stent structures the cross-sectional dimension of each stent structure within a zone can sequentially increase toward the outlet zone 410. One aspect of increasing the cross-sectional dimension toward the outlet zone 410 is to change properties of the stent structure. Increasing the cross-sectional dimension can both increase the radial force as well as be used to increase the amount of stent structure surface area contacting the vessel wall. This can be useful when arachnoid granulations are present. Arachnoid granulations typically occur in the lateral aspect of the transverse sinuses 120 and sometimes in the proximal sigmoid sinuses 110, as such, having more stent structure surface area against the vessel wall, per unit of area, in these regions can reduce localized stress concentrations. The medial transverse sinus 120 or superior sagittal sinus 130 are not as likely to have arachnoid granulations, and thus surface area can be less. The configuration of the stent structure can also be used to adjust the amount of stent structure surface area against the vessel wall, for example, by increasing the amount of material of the stent structure to reduce stress concentrations.

FIG. 2D is a side view schematically illustrating some embodiments of the stent assembly 300 in which the outlet zone 410 is reduced in diameter compared to one or more patency zones. The reduction in diameter of the outlet zone 410 may mitigate blood flow disturbances or turbulence out of the stent assembly 300 and into the native vessel. Additionally, a reduction in radial force in the outlet zone 410, with or without a reduction in diameter, as well as changing the conformal properties of the outlet zone 410, may be used to reduce/mitigate blood flow disturbances or turbulence.

FIGS. 2E-F are side views schematically illustrating some embodiments of the stent assembly 300 in which the ability of the stent structures to at least substantially approximate the native cross-sectional shapes of the native vessel is accomplished by changing the configuration and properties of the stent structure with or without changing the radial force between different stent structures or one or more zones. Stent structures or zones can have more struts 303 around the circumference than one or more other stent structures or zones. Increasing the number of struts 303 around the circumference can improve the ability of the stent structure to take the cross-sectional shape of the vessel in which it is deployed. For example, inlet zone 310 may have a similar number or more struts 303 than one or more transition zones 320/350, and one or more transition zones 320/350 may have similar number or more struts 303 than one or more patency zones 370/390. The outlet zone 410 may have a similar or more or less struts 303 than the one or more patency zones 370/390. Stent structure length (longitudinal) also can affect the ability of the stent structure to take the cross-sectional shape of the vessel in which it is deployed. The longitudinal length of stent structures (e.g., struts 303) can improve the ability of the stent structure to take the cross-sectional shape of the vessel in which it is deployed. Longer struts 303 (FIG. 2E) can provide improved conformance to the native vessel shape but reduce the longitudinal flexibility along a zone or the stent assembly 300 compared to shorter struts 303 (FIG. 2F) with the same material properties and cross-sectional dimensions. Shorter struts can provide a very gradual increase in radial force along a zone or region, high longitudinal flexibility, as well as different conformance properties along the length of the stent assembly 300. In addition, strut and link dimensions, properties (e.g., heat treating), etc., all influence the radial force and conformance to the native vessel. For example, the inlet zone 310 may have struts 303 with a similar or different length than those of one or more transition zones 320/350, and one or more transition zones 320/350 may have struts 303 with a similar or different length than those of one or more patency zones 370/390. The outlet zone 410 may struts 303 with a similar or different length than those of one or more patency zones 370/390.

FIG. 2G is a side cross-sectional view of some embodiments of a stent assembly 300. In this embodiment, the stent assembly includes the inlet zone 310, an optional first transition zone 330, an optional second transition zone 350, a first patency zone 370 (e.g., first body zone), an intermediate transition zone 360, a second patency zone 390 (e.g., second body zone), and an outlet zone 410 (not shown). Each zone can have one or more stent structures. For example, the inlet zone 310 can include an inlet stent structure 312, the first transition zone 330 can include a first transition stent structure 332, the second transition zone 350 can include a second transition stent structure 352, the first patency zone 370 can include a first patency stent structures 372, the intermediate transition zone 360 can include an intermediate transition stent structures 362, the second patency zone 390 can include a second patency stent structures 392, and the outlet zone 410 (not shown) can include an outlet stent structure 412 (not shown). Compared to the adjacent first/second patency stent structures 372/392, the intermediate transition stent structure 362 can have a smaller cross-sectional area (intermediate transition zone profile 364), lower radial force, different length struts, and/or other different features such that the intermediate transition stent structure 362 has different expansion and/or flexure properties. The number of zones and the number of stent structures in each zone can vary depending upon the application. Each stent structure can include at least one ring comprising struts 303 and/or at least one braided wire mesh. In some embodiments, the stent structures of at least some zones of the stent assembly 300 may have a round (e.g., circular) cross-sectional shape when unconstrained in free space (i.e., an unconstrained fully expanded state), and when deployed in the vasculature at least some of the stent structures of the stent assembly 300 may flex to at least generally approximate the relative shape(s) of the corresponding portions of the native vessel(s). In some embodiments, the stent structures of at least some zones of the stent assembly 300 may have the relative shape(s) of the corresponding portion(s) of the blood vessel(s) in an unconstrained fully expanded state and when deployed in the vasculature. In some embodiments, the stent structures of at least some zones of the stent assembly 300 may have the relative shape(s) of the corresponding portion(s) of the blood vessel(s) in an unconstrained fully expanded state and when deployed in the vasculature flex to further approximate the relative shape(s) of the corresponding portion(s) of the vessel(s). In some embodiments, the stent structures of at least some zones of the stent assembly 300 may have lower radial forces such that they do not expand the corresponding portion(s) of the blood vessel(s) in an unconstrained fully expanded state and when deployed in the vasculature flex to at least substantially approximate normal variances in the connection between the superior sagittal sinus 130 and the transverse sinus 120. The stent assembly 300 can also include interconnecting links 305 connecting adjacent stent structures together. Alternatively, adjacent stent structures may be directly connected to each other without the use of links 305. The number of links 305 connecting stent structures may vary along the length of the stent assembly 300.

FIG. 2H shows additional embodiment of the inlet zone 310 and the first patency zone 370. For example, the first patency zone 370 can have a radial force of approximately 0.001 N/mm to approximately 3 N/mm, and the inlet zone 310 can have a radial force of 0.001 N/mm to approximately 1.5 N/mm. The patency zone 370 can have a radial force near the rated diameter (e.g. 5, mm, 6 mm, 7 mm, 8 mm) of approximately 0.2 N/mm to 1.3 N/mm and in a more constrained state of for example 3.0 mm in diameter, a radial force of approximately 0.5 N/mm to 1.8 N/mm. The inlet zone 310 can have a radial force near the rated diameter (e.g. 5, mm, 6 mm, 7 mm, 8 mm) of approximately 0.1 N/mm to 1.2 N/mm and in a more constrained state of for example 3.0 mm in diameter, a radial force of approximately 0.3 N/mm to 1.5 N/mm. Illustrated are radiopaque markers 450 at each end of the stent assembly 300. The illustrated stent assembly 300 has 9 peaks and 3 links 305 per stent structure with 3 radiopaque markers 450 adjacent each end of the stent assembly 300. When deployed in the vessel, the patency zone 370 may have a generally circular cross-sectional shape 373 and the inlet zone 310 may have a generally triangular shape 314. In some embodiments, the stent assembly 300 can have only the inlet zone 310 and the first patency zone 370 shown and described with respect to FIG. 2H. In other embodiments, any of the stent assemblies 300 shown and described with respect to FIGS. 2A-2G can have the inlet zone 310 and first patency zone 370 shown and described with respect to FIG. 2H.

FIG. 2I shows an embodiment of a stent assembly 300 similar to that described in FIG. 2H, with the addition of radiopaque markers 450 adjacent the junction of the patency zone 370 and the inlet zone 310. The illustrated stent assembly 300 has 9 peaks and 3 links 305 per stent structure with 3 radiopaque markers 450 at each end of the stent assembly 300 and 3 radiopaque markers 450 adjacent the junction of the patency zone 370 and the inlet zone 310.

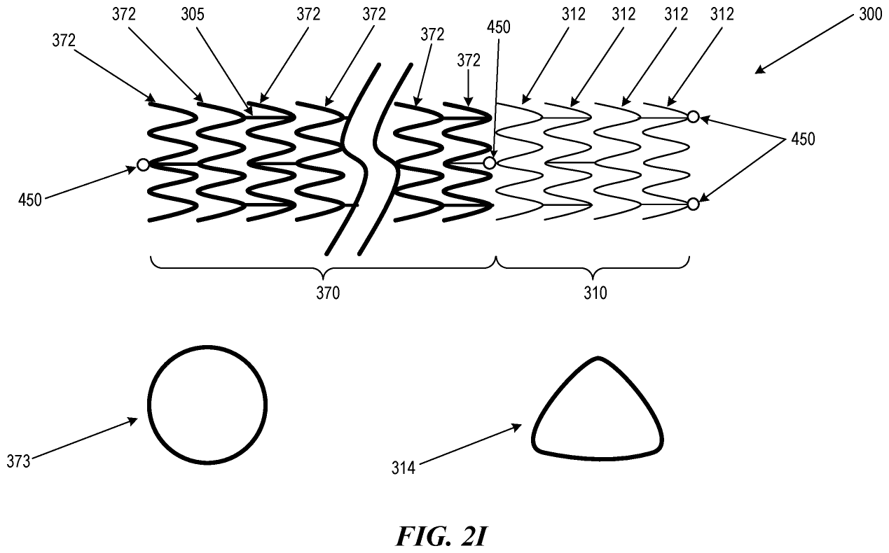

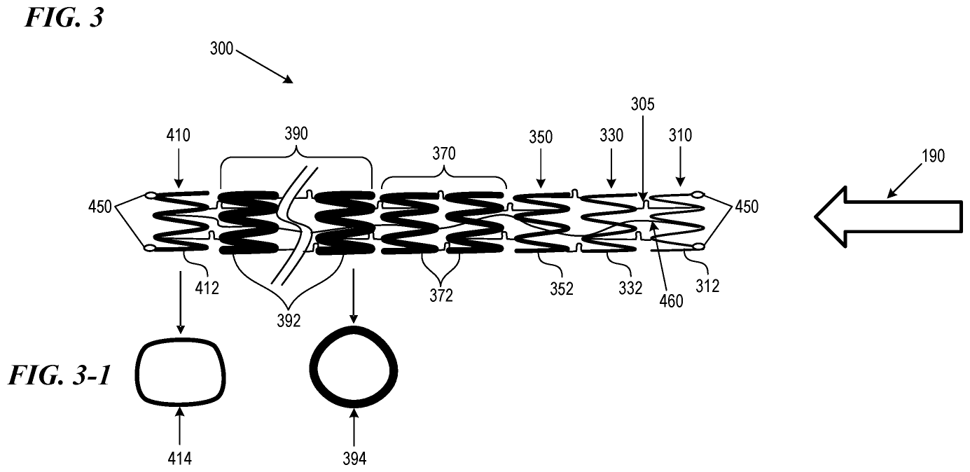

FIG. 3 is a side view schematically illustrating some embodiments of the outlet zone 410 of the stent assembly 300, and FIG. 3-1 is a cross-sectional view of the second patency stent structure 392 and the outlet stent structure 412. The outlet stent structure 412 can be configured direct the blood flow to have at least approximately the cross-sectional shape and dimensions of the vessel at the exit of stent assembly 300. Referring to FIG. 3-1, for example, the outlet stent structure 412 can have a rounded-quadrangle outlet shape 414 (e.g., a four-sided shaped with straight or curved sides and rounded corners) for at least substantially approximating the cross-sectional shape of the native sigmoid sinus 110. The outlet zone 410 can be similar to aspects of the inlet zone 310 and/or the transition zones (330/350), such as having one or more stent structures with transitions (not shown) of varying radial force and/or configuration (e.g., diameter and/or number and length of struts). The different radial forces and/or configuration along the stent assembly 300 from the inlet zone 310 to the outlet zone 410 provides a gradual transition that mitigates or avoids disruptions in the blood flow through the stented region and the adjacent regions.

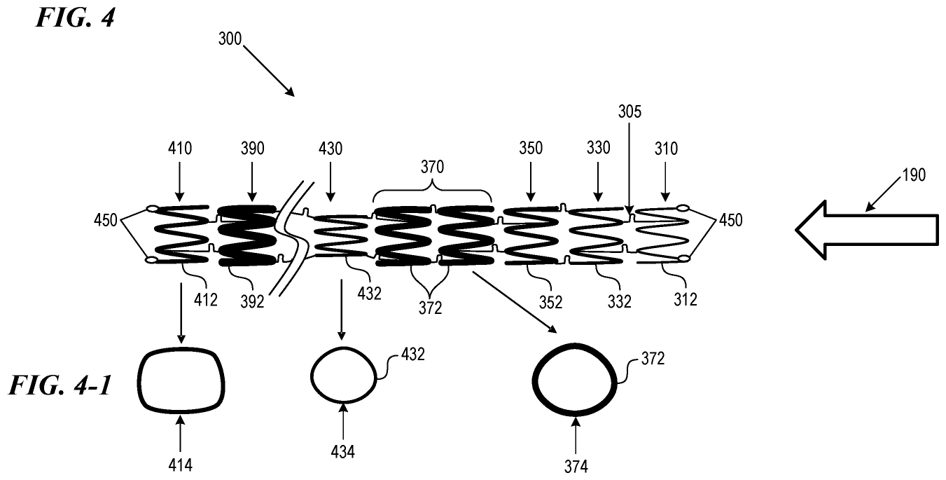

FIG. 4 is a side view schematically illustrating some embodiments of the stent assembly 300 with a transient pressure zone 430 having a transient stent structure 432, and FIG. 4-1 is a cross-sectional view of the patency stent structure 372, the outlet stent structure 412, and the transient stent structure 432. In some embodiments, the transient pressure zone 430 has one more transient stent structures 432, and each transient stent structure 432 has one or more radial forces and/or configuration (e.g., diameter and/or number and length of struts) that dynamically adapt to changes in the pressure of the cerebral spinal fluid. The transient pressure zone 430 accordingly enables the corresponding portion of the native vessel to accommodate rapid, transient increases in cerebral spinal fluid pressure. For example, a region within the transverse sinus 120 (FIG. 1) allows for a reduction in vessel cross-sectional area during normal physiologic spikes in cerebral spinal fluid pressure (e.g., during coughing). By having a transient pressure zone 430 proximate to the first patency zone 370 and/or the second patency zone 390, the deployed stent assembly 300 can mimic the functionality of the native vessel within the stented portion of the vessel. The transient pressure zone 430 may have a radial force of approximately 0.0002 N/mm to approximately 1.5 N/mm, though other radial forces may be suitable. FIG. 4-1 shows an example of transient pressure zone profile 434 of the transient stent structure 432 during a temporary increase in cerebral spinal fluid pressure.

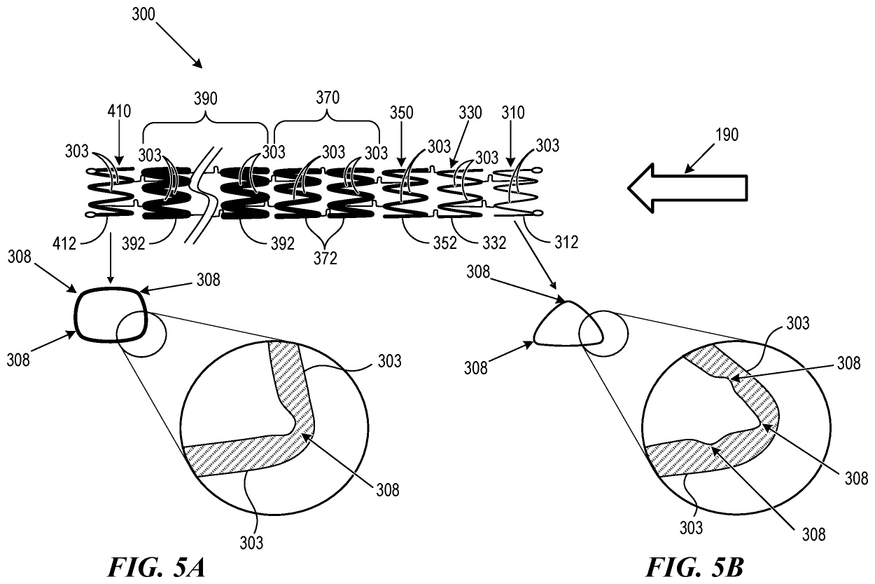

FIGS. 5A and 5B are detailed views of some embodiments of flex regions 308 along individual struts 303 of any of the embodiments of stent structures described herein. In the illustrated embodiment, FIG. 5A illustrates a single flex region 308 along struts 303 of the outlet stent structure 412 and FIG. 5B illustrates several flex regions 308 along struts 303 of the inlet stent structure 312. The flex regions 308 can be along any of the struts 303 of the other stent structures in any of the other zones, and a single stent structure can have several flex regions 308. The flex regions 308 enable the stent assembly 300 to preferentially flex at certain areas to better conform to the native vessel and thereby enhance the efficacy of the blood flow characteristics and simplify implanting the stent assembly 300. For example, the stent assembly 300 may have three or four flex regions 308 around the stent assembly 300 such that a deployed stent assembly 300 is able to better adapt to the shape of the native vessel. As a result, a single stent assembly 300 with sufficient flex regions 308 may be implanted anywhere from the superior sagittal sinus 130 to the sigmoid sinus 110, which would significantly simplify the process of stenting these vessels compared to implanting several individual stents. The flex regions 308 according enable the stent assembly 300 to both bend with the curvature of the vasculature (i.e., longitudinal flexure) and adapt to the cross-sectional shapes of the vasculature along the length of the stented region.

In any of the embodiments described herein, the stent structures can be coupled to adjacent stent structures with at least one link 305. The shape, dimensions, number and positioning of links 305 between stent structures may vary to obtain the desired properties of the stent assembly 300 (radial forces, longitudinal flexibility, resistance to longitudinal compression, delivery, etc.). For example, a high number of links 305 per stent structure (e.g., one link 305 for every one to three peaks or valleys of the stent structure) is expected to allow for recapture of the stent assembly 300 over that portion of the stent assembly 300. Whereas fewer links 305 (e.g., one link 305 every three or more peaks or valleys of the stent structure) improves the longitudinal flexibility of the stent assembly 300 to adapt to the curvature of the vessels during delivery and/after deployment.

In any of the embodiments disclosed herein, the links 305 may be straight, curved, zigzag, serpentine or other suitable shapes. The links 305 may have a bend for increased longitudinal flexibility as well as a reduction in longitudinal shortening of the stent assembly 300 upon deployment. The links 305 may have lengths from approximately 0.15 mm to approximately 20 mm or more, widths of approximately 0.0005″ to approximately 0.015″ or more, or 0.001″ to 0.010″, and thicknesses of approximately 0.0005″ to approximately 0.015″ or more, or 0.001″ to 0.010″, depending on the desired functionality. The number of links 305 between stent structures impacts the longitudinal flexibility of the stent assembly 300 as well as the area of coverage. A single link 305 connecting two adjacent stent structures can provide for significant longitudinal flexibility, whereas having a link 305 every few peaks/valleys of the stent structures (e.g., one link 305 every two to five peaks/valleys) provides additional coverage of the vessel and increases resistance to compression while maintaining some amount of flexibility. The number, shape and dimensions of the links 305 can be selected to balance the longitudinal flexibility requirements with the amount of coverage and the desire to recapture the stent assembly 300 in a delivery catheter.

In certain embodiments, the links 305 may be arranged in peak-to-valley, peak-to-peak, peak-to-side, valley-to-side, crisscross, boxcar or other suitable configurations. A peak-to-valley configuration is a configuration of the design that reduces the amount of longitudinal shortening when the stent assembly 300 is deployed, including a peak-to-valley configuration where the peak and valley are not longitudinally aligned along the stent assembly 300. In some embodiments, all or a portion of the stent assembly 300 may be recaptured by connecting links 305 to each peak of a strut-type stent structure with a corresponding peak of an adjacent strut-type stent structure in a mesh configuration, boxcar configuration, or other suitable configuration.

In any of the embodiments disclosed herein, at least some of the stent structures can be directly connected to each other using welds, an adhesive, sutures or other suitable fasteners. For example, any of the stent structures 312, 332, 352, 372, 392, 412 and/or 432 can be directly connected to an adjacent stent structure. In some embodiments, only some of the stent structures may be directed connected to each other using a weld, adhesive, suture other fastener, while other of the stent structures are attached to adjacent stent structures using links 305.

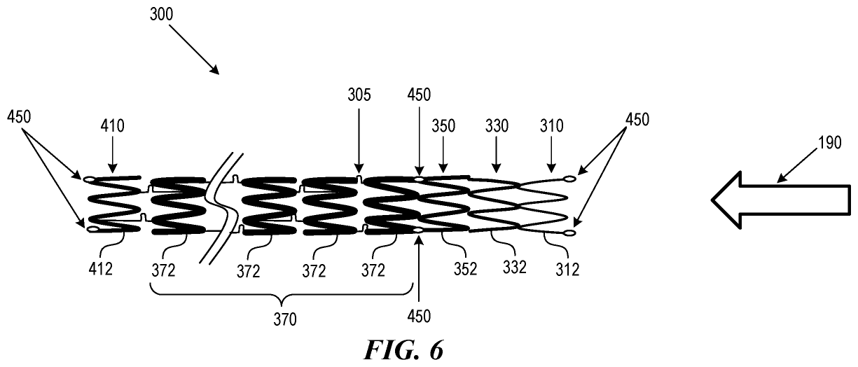

FIG. 6 is a side view schematically illustrating a stent assembly 300 in which stent structures 312, 332 and 352 of the zones 310, 330, and 350, respectively, are directly connected to each other without the having links 305, and the stent structures 372 and 412 forming zones 370 and 410, respectively, are connected to each other with links 305. This configuration is useful for being able to recapture a partially deployed stent assembly 300. For example, the stent assembly 300 can be partially deployed from a delivery catheter and recaptured within the delivery catheter if the inlet zone 310 and/or transition zones 330/350 are not positioned correctly to locate the patency zone 370 at the narrowed portion of the vessel or if there is otherwise insufficient relief of symptoms. Any number of stent structures can be directly connected to each other such that a desired length of the stent assembly 300 can be partially deployed and still be recaptured for repositioning or removal. As previously described, a suitable number of links 305 can also be used to enable recapture of a partially or fully deployed stent assembly 300.

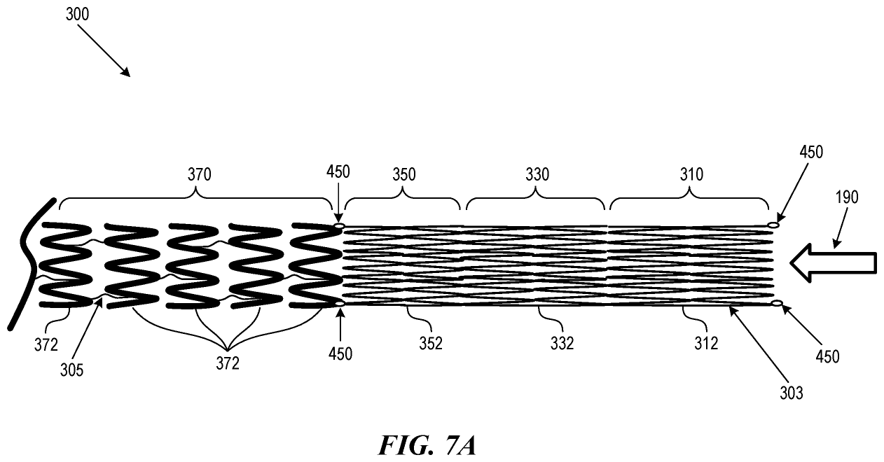

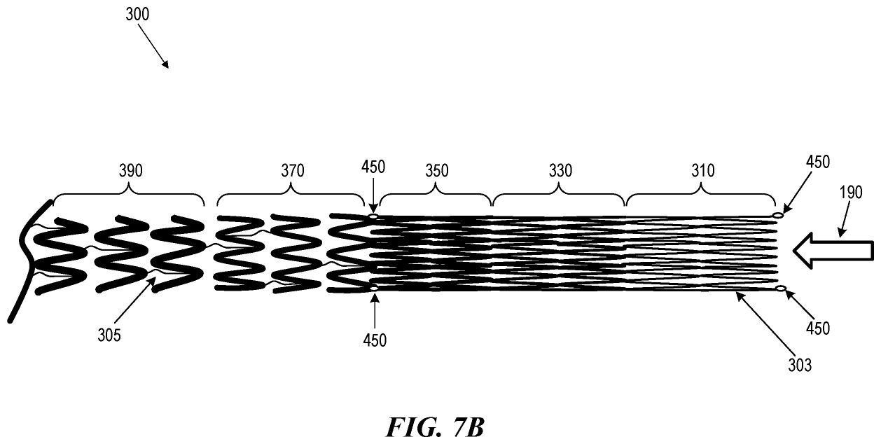

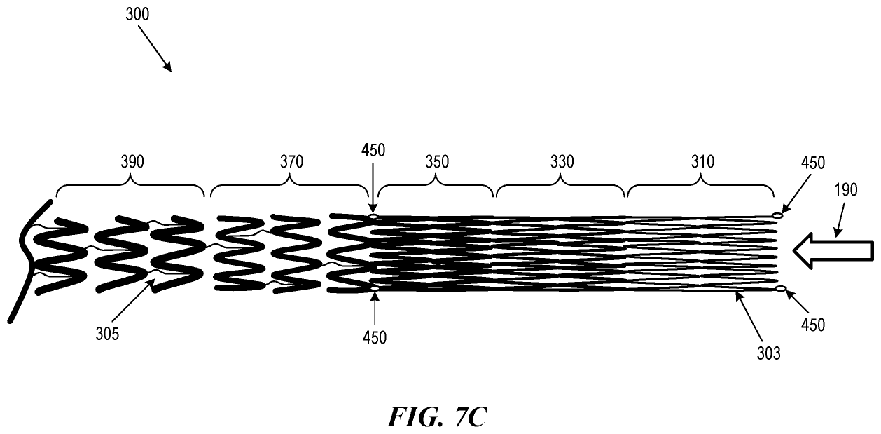

FIGS. 7A-C are side views schematically showing stent assemblies 300 with stent structures having selected lengths or with struts 303 having selected thicknesses that can be applied to any embodiments disclosed herein. In some embodiments, the length of the stent structures may vary in one or more zones to optimize the stent assembly 300 for specific anatomical features. For example, the lengths of the stent structures in some zones can vary to accommodate the shape as well as more tortuous regions of the anatomy. This is expected to improve the interface with the vasculature and thereby enhance blood flow characteristics and navigability through the anatomy. FIGS. 7A and 7B show some embodiments in which the stent structures 312, 332 and 352 of the inlet zone 310, first transition zone 330 and the second transition zone 350, respectively, are longer than the stent structures 372 of the first patency zone 370 or the stent structures 392 of the second patency zone 390 (FIG. 7B only). The longer stent structures and/or greater number of circumferential struts 303 in the inlet zone 310 and the transition zones 330/350 enable the stent assembly 300 to at least substantially approximate the native cross-sectional shapes of the native vessel. Conversely, as shown in FIG. 7C, the stent structures of the inlet zone 310 and/or the transition zones 330/350 can be shorter than those of the patency zones 370/390 to provide a very gradual change of cross-sectional profile from the native vessel into the stent assembly 300 and through the patency zones. The outlet zone 410 may also be longer or shorter and vary in number of struts 303 than the patency zones 370/390 for smooth outflow of blood from the stent assembly 300. The longitudinal length of the stent structures, when constrained, may be from approximately 0.25 mm to approximately 5 mm for relatively shorter stent structures and from approximately 0.5 mm to approximately 10 mm for relatively longer stent structures.

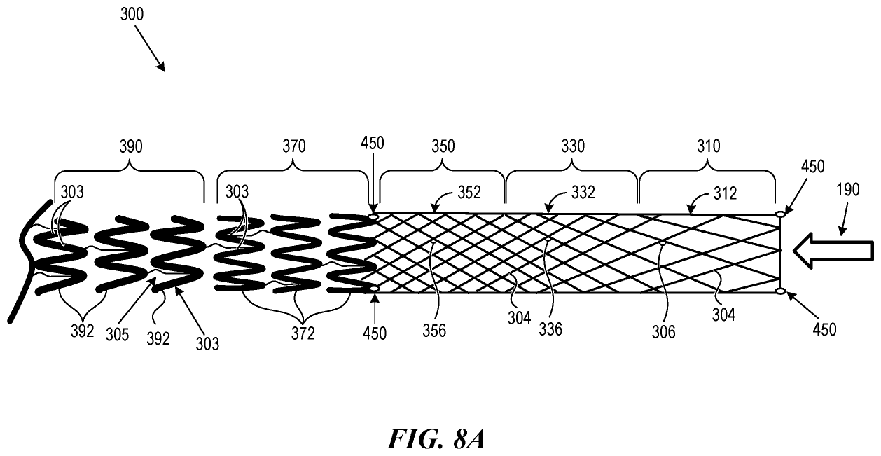

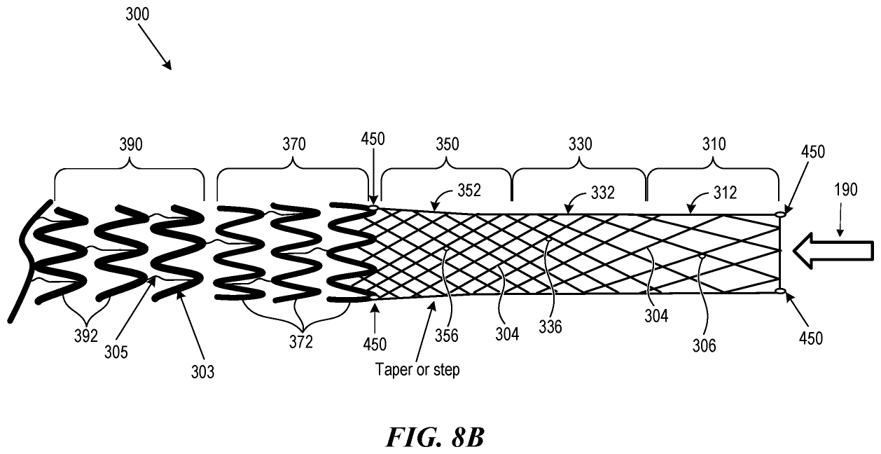

FIGS. 8A and 8B are side cross-sectional views illustrating stent assemblies 300 in which some zones comprise braided filaments 304 and some zones comprise cut or etched structures with struts 303. In some embodiments, the stent assembly 300 may be, but is not limited to, a mesh, a cut or etched material, a braided material, or any combination of structures to form a non-solid-walled structure. In some embodiments, the filaments 304 comprise metallic wires (e.g., Nitinol or stainless steel wires), polymeric strands, or other suitable materials, and a combination thereof. The braid angles and pore sizes between the filaments along with the filament density and filament thickness can be selected for each of the stent structures along the stent assemblies 300 to provide the desired radial forces, shapes, and flexibility for each zone.

Referring to FIG. 8A, the inlet zone 310 has an inlet stent structure 312 and the transition zone 330 has a transition stent structure 332 that may comprise a braided mesh having wire filaments 304 configured to have a generally circular cross-sectional shape in an unconstrained expanded state and to flex such that stent structures 312 and 332 at least substantially approximate the shape of the corresponding region of the vessel where they are implanted. In some embodiments, such as when implanting the inlet zone 310 and/or the first transition zone 330 in the superior sagittal sinus 130 (FIG. 1) or the transverse sinus 120, the inlet zone 310 and the first transition zone 330 flex to have a three-sided cross-sectional shape (e.g., at least generally triangular) that at least substantially approximates the cross-sectional shapes of the superior sagittal sinus 130 and/or the transverse sinus 120. In some embodiments, the inlet zone 310 and the first transition zone 330 have a three-sided shape (e.g., at least generally triangular) in an unconstrained expanded shape and can also flex to at least substantially approximate a generally triangular cross-sectional shape of the vessel in which they are implanted.

Referring still to FIG. 8A, the inlet stent structure 312 may have a lower radial force and/or otherwise be configured to be more conformable to the cross-sectional shape of the native vessel than the transition stent structure 332. For example, the filaments 304 along the inlet stent structure 312 may have an inlet braid angle 306 and the filaments 304 along the transition stent structure 332 may have a transition braid angle 336 less than the inlet braid angle 306. As a result, the transition stent structure 332 has smaller pore sizes and can have higher radial force than the inlet stent structure 312. In some embodiments, addition filaments 304 may be woven into the braid along the transitions stent structure 332 to impart a greater radial force and/or density than the inlet stent structure 312. In some embodiments, the stent assembly can include an optional second transition zone 350 with a second transition stent structure 352 comprising filaments 304 braided at a second transition braid angle 356 less than the transition braid angle 336. The second transition stent structure 352 may accordingly have a greater radial force and/or density than the first transition stent structure 332. The second transition stent structure 352 can be attached to the first patency stent structure 372 of the first patency zone 370, and as explained above the first patency stent structure 372 can comprise struts 303 formed from a cut or etched hypo-tube. As with other embodiment, the stent assembly 300 can comprise an optional second patency stent structure 390 defining a second patency zone 390, an optional transient stent structure 432 defining an optional transient pressure zone 430 (not shown), and/or an optional outlet stent structure 412 defining an optional outlet zone 410 (not shown).

Referring to FIG. 8B, one or more of the stent structures 312/332, and optionally stent structure 352, can increase in cross-sectional area in a downstream direction to the first patency stent structure 372 of the first patency zone 370. For example, in one embodiment, the diameter of the stent assembly 300 can increase gradually throughout the stent structures 312/332 and optionally stent structure 352. In other embodiments, the inlet stent structure 312 can have at least a substantially constant cross-sectional dimension in an unconstrained expanded state and the cross-sectional dimension of one or both the first transition stent structure 332 and the second transition stent structure 352 can increase to the first patency stent structure 372. The stent assembly 300 may be fabricated to be inherently round (e.g., made from a round tubing or mesh) when loaded in a delivery catheter and then at least one or more portions of the stent assembly 300 at least partially conforms to the vessel wall when deployed. The stent assembly 300 may also be fabricated to be inherently non-round in the deployed and/or collapsed state, e.g., made non-round, such as a three-sided (e.g., generally triangular) and/or four-sided (e.g., rounded quadrangle) tube or mesh.

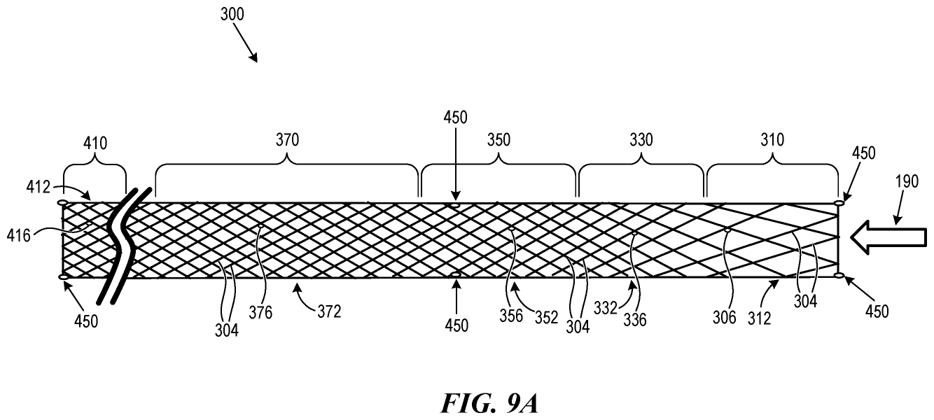

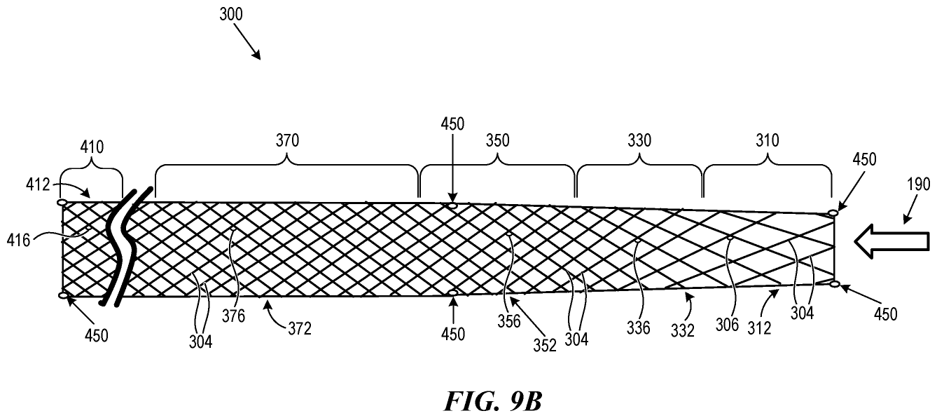

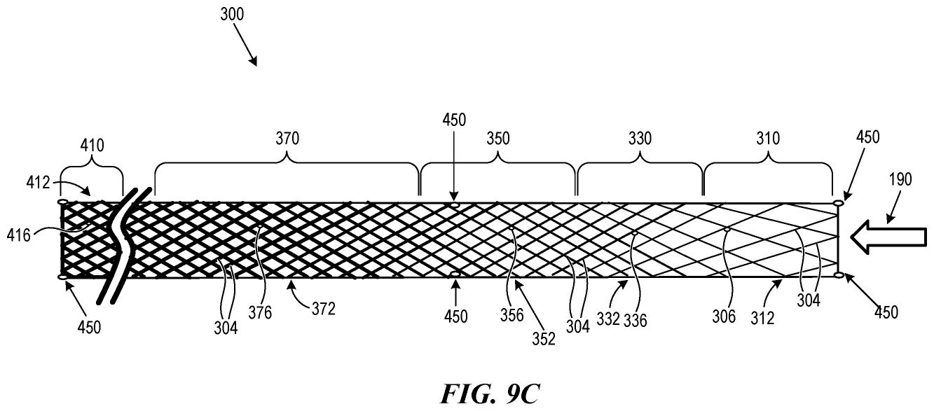

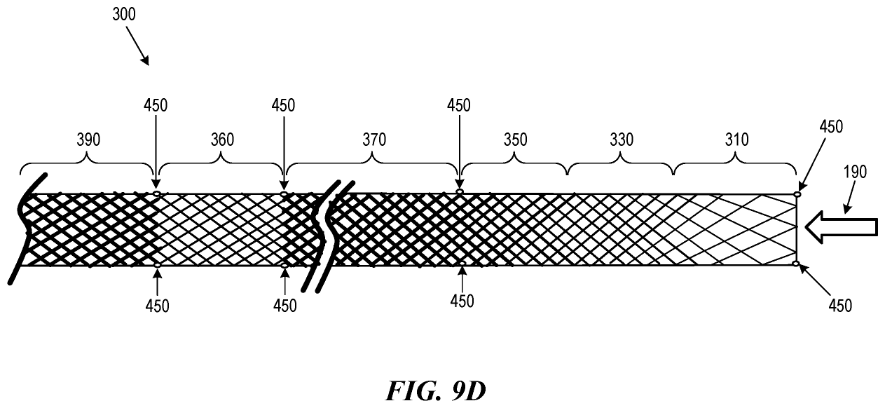

FIGS. 9A-9D are side views schematically illustrating stent assemblies 300 that comprise braided filaments 304 from the inlet to the outlet. In some embodiments, the filaments 304 comprise metallic wires (e.g., Nitinol or stainless steel wires), polymeric strands, or other suitable materials and a combination thereof. The braid angles and pore sizes between the filaments along with the filament density and filament thickness can be selected for each of the stent structures along the stent assemblies 300 to provide the desired radial forces, density, shape, ability to at least substantially conform to the native vessel, and flexibility for each zone. In some embodiments, for example, the stent assembly 300 may be a braided design with variable radial forces along the length of the stent assembly 300 obtained by altering the braid angle along the length of the stent assembly 300. The inlet portion of the stent assembly 300 may have a larger braid angle, which can result in a lower radial force and is expected to allow the inlet portion to be more conformal. In certain embodiments, the stent assembly 300 may be a braided design that has one or more strands added along its length to increase the radial force or density in certain sections. In certain embodiments, the stent assembly 300 may be a braided design that has one or more thicker strands of material woven into certain sections of the stent assembly 300 to obtain variable radial force.

Referring to FIG. 9A, in some embodiments the braid angles of the filaments 304 decreases, which decreases the pore size between filaments 304, from the inlet to the outlet of the stent assembly 300. For example, in the illustrated embodiment, the inlet stent structure 312 has an average braid angle 306 larger than an average braid angle 336 of the first transition stent structure 332, and the average braid angle 306 is larger than the average braid angle 356 of the second transition stent structure 352. Similarly, the average braid angle 356 can be larger than the average braid angle 376 of the first patency zone 370. The braid angle in each stent structure can be at least substantially constant through each stent structure, or the brain angle can change along at least a portion of each stent structure. In some embodiments, a braid angle 416 of the outlet stent structure 412 is the same or larger than the braid angle 376 of the patency stent structure 372 (shown), but in other embodiments the braid angle 416 is greater than the braid angle 376 such that the pore size of the outlet stent structure 412 is greater than the patency stent structure 372. For example, the braid angle 376 can be about the same as the braid angle 336 or the braid angle 306.

Referring to FIG. 9B, in some embodiments the cross-sectional dimension of the stent assembly 300 can change along the length of the braided material. For example, the braided material in the inlet zone 310 can have a cross-sectional dimension that is less than that of the braided material in the adjacent transition zone(s) 330/(350). The cross-sectional dimension can change gradually in some of all of the zones, or the cross-sectional dimension can be constant throughout some zones. For example, the cross-section dimension can gradually increase throughout the inlet zone 310 and the transition zone(s) 330/(350) and then remain at least substantially constant throughout the patency zone 370

Referring to FIG. 9C, in some embodiments the thickness of the filaments 304 can be different in different zones. For example, the filaments 304 in the inlet zone 310 can be thinner than those in downstream zones 330, 350 and 370. The thickness of the filaments 304 can be at least substantially constant throughout a zone, or the thickness of the filaments 304 can gradually change along a zone or along the length of several zones. As shown in FIG. 9C, the thickness of the filaments 304 can increase and the braid angle can decrease in the downstream direction from one zone to the next. This combination enhances the flexibility of the inlet zone 310 to adapt to the cross-sectional shape of the native vessel so that disruptions to the inlet flow are mitigated or eliminated, which is expected to reduce or eliminate revisions, while the patency zone 370 can have sufficient radial force to overcome the resistance of the native vessel wall and cerebral spinal fluid pressure at the narrowed portion of the vessel for restoring patency

Referring to FIG. 9D, any of the above embodiments can be applied to a stent assembly 300 which has an intermediate transition zone 360 located between a first patency zone 370 and a second patency zone 390. This configuration is especially useful for a stent assembly 300 that spans from the superior sagittal sinus 130 to the sigmoid sinus 110. The stent assembly 300 may have a first patency zone 370 located at least partially within the superior sagittal sinus 130 and a second patency zone 390 located at least partially within the transverse sinus 120 and can extend into the sigmoid sinus 110. The one or more patency zones may be directly connected or may include an intermediate transition zone 360 and/or transient pressure zone 430.

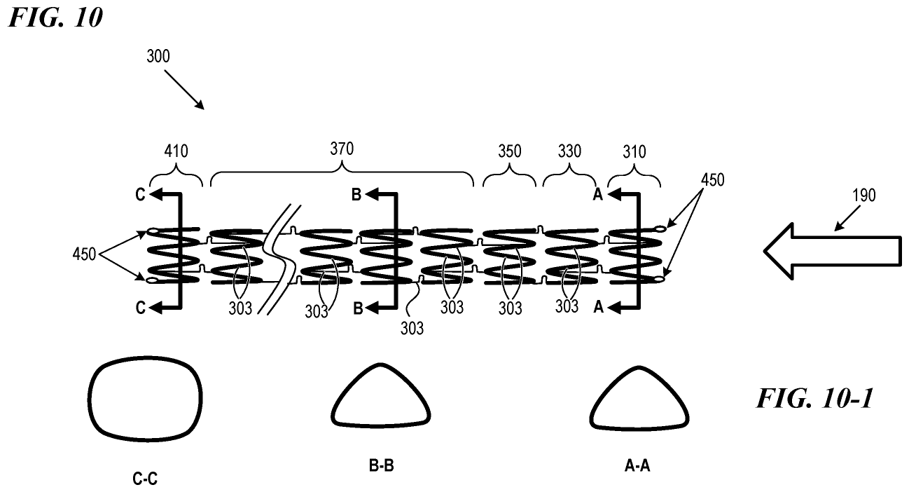

FIG. 10 is a side view schematically illustrating a stent assembly 300 having non-circular cross-sectional shapes in an unconstrained fully expanded state and/or as deployed in a blood vessel. The stent assembly 300 can have three-sided cross-sectional shapes (e.g., relatively triangular shapes) at the inlet zone 310 (cross-section A-A) and through one or more patency zones (e.g., patency zone 370) (cross-section B-B) and a four-sided shape (e.g., a rounded quadrangle) in one or more patency zones (e.g., patency zone 390) and at the outlet zone 410 (cross-section C-C). The stent assembly 300 may have a combination of shapes in the collapsed and deployed state, such as triangular in one region and quadrangle in another. In addition to cross-sectional shapes, the cross-section sizes may change along the length of the stent assembly 300 as well. For example, a non-round stent assembly 300 configured for stenting the transverse sinus 120 and the sigmoid sinus 110 can cover the native vasculature from at or adjacent the torcula and/or from within the superior sagittal sinus 130 and extend from there into the sigmoid sinus 110. Such a stent assembly 300 may be fabricated by cutting or etching struts 303 from round tubing with a varying diameter to account for the vessel perimeter.

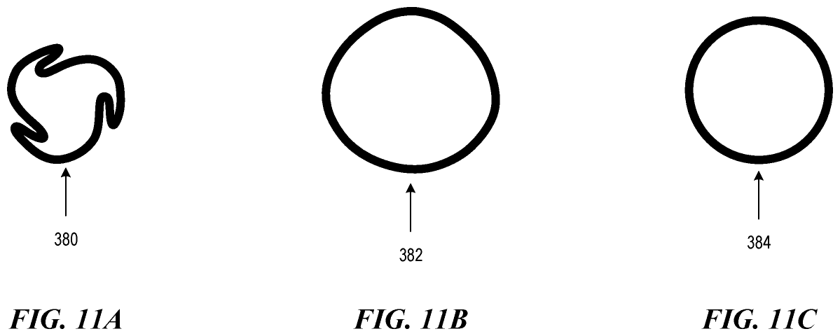

FIGS. 11A-11C are cross-sectional views illustrating different cross-sectional shapes for a portion of a selected stent structures. At least a portion of the stent assembly 300 may be constructed with a first configuration, stent structure overlap 380, while constrained within the delivery catheter 200 (FIG. 11A). After deployment, the stent assembly 300 may expand to a larger perimeter, stent structure expanded 382 (FIG. 11B). After expansion to the stent structure expanded 382 perimeter, any external forces on the stent assembly 300 can only compress (e.g., reduce the perimeter) the stent assembly 300 (stent structure recompressed 384) to a perimeter that is greater than the stent structure overlap 380 perimeter and less than the stent structure expanded 382 perimeter (FIG. 11C). This configuration provides for a stent assembly 300 that is low profile during deployment, expands to a desired perimeter, and maintains a certain patency (minimum perimeter) under extreme physiologic conditions. For example, in the case of a stent assembly 300 having a pre-deployment perimeter of 3.8 mm when loaded in a delivery catheter, the stent assembly 300 can have a minimum deployed perimeter of not less than approximately 9 mm to maintain the equivalent flow area of approximately a 3 mm diameter tube. This may be accomplished by having radial sections of the stent structure(s) overlap 380 while in the delivery catheter 200, and upon being deployed (stent structure expanded 382) the stent structure(s) do not overlap. As such, after deployment, compression (stent structure recompressed 384) of the stent structure(s) does not allow that overlapping to reoccur, which maintains a minimum cross-sectional area that is larger than when constrained within the delivery catheter 200.

As explained above, in any of the embodiments described herein the stent structures and links 305 may be different at various locations to adjust the radial force, density, shape, ability to at least substantially conform to the native vessel, and flexibility. The inlet zone 310 typically has the lowest radial force and/or greatest ability to at least substantially conform to the native vessel, transition zones 330/350 have higher radial forces and relatively less ability to at least substantially conform to the native vessel, and patency zones 370/390 have still higher radial forces and even less ability to at least substantially conform to the native vessel. At least a portion of a second patency zone 390 typically has a larger perimeter than a portion of a first patency zone 370 in the deployed state within the vessel, and the change in perimeter can be gradual.

In any of the foregoing embodiments, the number struts 303 per stent structure, and/or the width and/or the thickness of the struts 303 can be selected to accommodate the desired radial force, density, shape, ability to at least substantially conform to the native vessel, flexibility, and dimensions of the stent structure. The struts 303 may have widths from approximately 0.0005″ to approximately 0.015″ or more, and the struts 303 may have thicknesses from approximately 0.0005″ to approximately 0.015″ or more. The dimensions of the struts 303 may change within a single stent structure, e.g., the struts 303 on one longitudinal side of the stent structure may be different than those on the other longitudinal side of the stent structure.

In any of the embodiments disclosed herein, the stent assembly 300 may comprise a self-expanding, shape memory material, including NiTi or an NiTi alloy (e.g., Ni, Ti, and Cr). Other suitable materials include stainless steel and biocompatible polymeric materials having a desired elasticity and spring forces.

In any of the embodiments disclosed herein, the stent assembly 300 may include one or more radiopaque markers 450 to enhance visualization of the stent assembly 300 and/or regions of the stent assembly 300 (e.g., ends, patency zone(s), transition zone(s), outlet zone, etc.) before, during, and/or after placement. The radiopaque markers 450 can be made from but are not limited to tantalum, platinum, iridium, gold, tungsten, zirconium, or any combination or alloys thereof. The radiopaque markers 450 may be attached to the stent assembly 300 by being bonded, swagged, crimped, wrapped, welded, weaved, or any method of affixing the radiopaque marker 450. An example radiopaque marker 450 construction is to form an opening in the stent structure, such as a relatively round opening and swage or crimp a radiopaque material (e.g., gold, tantalum) into the opening. The radiopaque marker 450 can be a coating applied to selected portions of the stent assembly, or as shown in FIG. 3 the radiopaque marker 450 can be one or more radiopaque strands 460 incorporated into any of the stent assemblies 300 shown in FIGS. 2A-10 such that operators can visualize one or more portions and/or the full length of the stent assembly 300.

In any of the embodiments disclosed herein, at least a portion of the stent assembly 300 may be coated with materials to enhance the efficacy and or deliverability of the stent assembly 300. For example, such coating may include anti-thrombogenic, anti-platelet, and/or anti-cell proliferation coatings to minimize or eliminate thrombus formation and tissue build up. Other coatings can include surface modifiers to change the surface properties of the stent assembly 300, such as to increase or decrease lubricity/friction to aid in stent assembly 300 delivery, deployment, recapture, placement, and the like.

In any of the embodiments described herein, at least a portion of the stent assembly 300 may be constructed such that after deployment, there is a minimum perimeter (or diameter) to which all or at least that portion of the stent assembly 300 can be recompressed. As such, all or at least that portion of the stent assembly 300 is prevented from compressing (collapsing) below a cross-sectional area that would cause symptoms to reoccur from high cerebral spinal fluid pressure. Such a configuration provides for a stent assembly 300 that is low profile during deployment, expands to a desired perimeter, and maintains a certain patency (minimum perimeter) under extreme physiologic conditions. For example, in the case of a stent assembly 300 having a pre-deployment perimeter of 3.8 mm when loaded in a delivery catheter, the stent assembly 300 can have a minimum deployed perimeter of not less than approximately 9 mm to maintain the equivalent flow area of approximately a 3 mm diameter tube, which generally will reduce and/or eliminate symptoms.

In any of the embodiments described herein, the stent assembly 300 may be constructed such that upon partial or full deployment, the stent assembly 300 can be recaptured within a delivery catheter. For example, the stent assembly 300 can be in the vessel and at least partially deployed, and then the patient can be assessed for blood flow, resolution of tinnitus (e.g., pulsatile tinnitus), or other symptoms. If the results are not satisfactory, the stent assembly 300 can be recaptured and removed or repositioned at a different location in the vasculature. If repositioned, the patient can be reassessed for efficacy of the new implant location. Additionally, if stenting does not achieve the desired outcome after positioning or repositioning, the stent assembly 300 can be removed from the patient. When this occurs, the patient does not have to follow the medical requirements post-stent assembly 300 implantation and does not have a permanent implant (stent assembly 300).

In any of the embodiments described herein, the perimeter of the stent structures is sized to provide the desired collapsed diameter of the stent assembly 300 and also the desired deployed perimeter. The perimeter is used instead of diameter when at least a portion of the deployed stent assembly 300 is not round in cross-section within the vessel because it may be more useful to determine the perimeter of the deployed stent assembly 300 and match that to the perimeter of the native vessel than matching a diameter of a circular shape. The perimeter may be under- or over-sized as determined by the doctor. When deployed, the perimeter may be consistent along the length of the stent assembly 300, or it can vary. For example, the perimeter can vary among segments or zones, and may have transitions or be partially or continuously variable. When collapsed for loading into/onto a delivery catheter or wire, the perimeter of the stent structures may be less than or equal to 6 F, or 4 F, or even smaller. For example, the perimeter of a collapsed stent structure can be less than or equal to approximately 5.8 mm or less than or equal to approximately 4.0 mm. The perimeter of a deployed stent structure depends on the target vessel. When the stent assembly 300 is deployed from adjacent or at the torcula, or in the superior sagittal sinus 130, and extends into the sigmoid sinus 110, the range of sizes is such that the deployed stent assembly 300 has a perimeter of approximately 5 mm to approximately 40 mm, or from approximately 9 mm to approximately 35 mm, which as previously stated can vary along the length of the stent assembly 300. Various perimeters can be made available to allow for varying patient vessel sizes, such as a series of sizes with perimeters of approximately 16 mm, 19 mm, 22 mm, and 25 mm.

The lengths of the stent structures as measured along the longitudinal axis of the stent assembly 300 are sized to provide for the desired compressed and deployed perimeters as well as the required flexibility to navigate the anatomy to the target location. The lengths of the stent structures along the stent assembly 300 may vary to account for different parameters, such as radial force, area of coverage, the ability of the stent structure to take the cross-sectional shape of the vessel, etc.

In any of the embodiments described herein, the stent assembly 300 can be tailored for and deployed within a specific vasculature. For example, in some embodiments a stent assembly 300 configured for stenting the transverse sinus 120 and sigmoid sinus 110 can be implanted in the vasculature from at or adjacent the torcula, such as within the superior sagittal sinus 130, and extend to the sigmoid sinus 110. In some embodiments, a stent assembly 300 from the torcula into the sigmoid sinus 110 would have a length of approximately 50 mm to approximately 120 mm or approximately 60 mm to 100 mm. In some embodiments, a stent assembly 300 for implanting from within the superior sagittal sinus 130 into the sigmoid sinus 110 would have a length of approximately 80 mm to 300 mm or approximately 100 mm to 180 mm. In some embodiments, a stent assembly 300 configured specifically for being implanted in one of the transverse sinuses 120 only or the sigmoid sinus 110 only would have length of approximately 20 mm to approximately 80 mm, or from approximately 30 mm to 50 mm. In some embodiments, a stent assembly 300 specifically configured for being implanted in the superior sagittal sinus 130 only may have a typical length of approximately 20 mm to approximately 200 mm, or from approximately 40 mm to approximately 100 mm. In some embodiments, a stent assembly 300 for being implanted in the superior sagittal sinus 130 is typically triangular in cross-section (like the transverse sinus 120) with a typical perimeter of approximately 5 mm to approximately 40 mm, or from approximately 9 mm to 26 mm. Various perimeters can be made available to allow for varying patient vessel sizes, such as a series of sizes with perimeters of approximately 16 mm, 19 mm, 22 mm, and 25 mm.

In some embodiments, an example configuration of the stent assembly 300 for use in the transverse sinus 120 and extending into the sigmoid sinus 110 is as follows: the stent assembly 300 is constructed of NiTi and includes stent structures and links 305; the inlet zone 310 extends for a nominal length of approximately 3 mm to 15 mm, 5 mm to 12 mm (e.g., 10 mm), or 3 mm to 5 mm, and has a radial force near the rated diameter (e.g. 5, mm, 6 mm, 8 mm) of approximately 0.1 N/mm to 1.2 N/mm and in a more constrained state of 3.0 mm in diameter, a radial force of approximately 0.3 N/mm to 1.5 N/mm, with stent structure 312 longitudinal lengths of approximately 0.15 mm to approximately 5 mm or approximately 2.3 mm and link 305 lengths of approximately 0.18 mm to approximately 6 mm or approximately 2.8 mm. The perimeter of the inlet zone 310 can be approximately 16 mm to 26 mm. Various perimeters can be made available to allow for varying patient vessel sizes, such as a series of sizes with perimeters of approximately 16 mm, 19 mm, 22 mm, and 25 mm.

In any of the embodiments described herein, the stent assembly 300 can have first transition zone 330 which extends for a nominal length of approximately 3 mm to 10 mm or 5 mm and has a radial force of approximately 0.001 N/mm to 2 N/mm, or approximately 0.6 N/mm at the stent structure 332 adjacent the inlet zone 310 and may increase throughout the first transition zone 330 to approximately 0.5 N/mm to 1 N/mm, or approximately 0.4 N/mm. Such a first transition zone 330 can have stent structure 312 longitudinal lengths of approximately 0.15 mm to approximately 5 mm or approximately 2.3 mm and link 305 lengths of approximately 0.18 mm to approximately 6 mm or 0.18 mm to 2.8 mm. The perimeter of the inlet zone 310 can be approximately 16 mm to 26 mm. The perimeter of a first transition zone 330 can be approximately 16 mm to approximately 26 mm. Various perimeters can be made available to allow for varying patient vessel sizes, such as a series of sizes with perimeters of approximately 16 mm, 19 mm, 22 mm, and 25 mm.

In any of the embodiments described herein, a stent assembly 300 can have a second transition zone 350 which extends for a nominal length of approximately 0.3 mm to 10 mm, or 5 mm and has a radial force from approximately 0.002 N/mm to 2.5 N/mm or approximately 0.6 N/mm at the stent structure 352 adjacent the first transition zone 330. The radial force can increase throughout the second transition zone 350 to approximately 1 N/mm to 3 N/mm, or approximately 0.8 N/mm. The second transition zone can have a stent structure 352 longitudinal length of approximately 0.15 mm to approximately 5 mm or 0.015 mm to 2.3 mm and link 305 lengths of approximately 0.18 mm to approximately 6 mm or 0.18 mm to 2.8 mm. The perimeter of a second transition zone 350 can be approximately 16 mm to approximately 26 mm. Various perimeters can be made available to allow for varying patient vessel sizes, such as a series of sizes with perimeters of approximately 16 mm, 19 mm, 22 mm, and 25 mm.

In any of the embodiments described herein, the first patency zone 370 can extend for a nominal length of approximately 20 mm to 160 mm or 90 mm and have a radial force near the rated diameter (e.g. 5, mm, 6 mm, 8 mm) of approximately 0.2 N/mm to 1.2 N/mm and in a more constrained state of 3.0 mm in diameter, a radial force of approximately 0.5 N/mm to 1.8 N/mm. The stent structure 372 of the first patency zone 370 can have a length of approximately 0.3 mm to approximately 5 mm or approximately 2.5 mm and link 305 lengths of approximately 0.35 mm to approximately 6 mm or approximately 2.7 mm. The perimeter of the first patency zone 370 can be approximately 16 mm to approximately 26 mm. Various perimeters can be made available to allow for varying patient vessel sizes, such as a series of sizes with perimeters of approximately 16 mm, 19 mm, 22 mm, and 25 mm.

In any of the embodiments described herein, the stent assembly 300 can have an outlet zone 410 which extends for a length of approximately 3 mm to 15 mm, or 8 mm and has a radial force of 0.05 N/mm to 3 N/mm, or approximately 0.8 N/mm, at an upstream end (e.g., adjacent to the first patency zone 370 or the second patency zone 390), and the radial force of the outlet zone 410 decreases throughout the outlet zone 410 to approximately 0.05 N/mm to 2 N/mm or approximately 0.6 N/mm. The stent structure 412 of the outlet zone 410 can have a length of approximately 0.15 mm to approximately 5 mm or approximately 2.3 mm and link 305 lengths of approximately 0.15 mm to approximately 6 mm or approximately 2.8 mm, and a perimeter of approximately 16 mm to approximately 28 mm. Various perimeters can be made available to allow for varying patient vessel sizes, such as a series of sizes with perimeters of approximately 16 mm, 19 mm, 22 mm and 25 mm.

In any of the embodiments described herein, a stent assembly 300 for placement in the contralateral (non-dominant) transverse sinus 120, and optionally at least in part into the sigmoid sinus 110, is typically smaller in perimeter than when placed relative to the dominant transverse sinus 120 of the same patient. An example of a stent assembly 300 for use in the contralateral (non-dominant) transverse sinus 120 can be constructed as previously described, with a patency zone 370 length of approximately 20 mm to 60 mm or approximately 40 mm and a perimeter of the inlet zone 310 can be approximately 9 mm to approximately 19 mm. Various perimeters can be made available to allow for varying patient vessel sizes, such as a series of sizes with perimeters of approximately 9 mm, 12 mm, 16 mm, and 19 mm.

An example configuration of a stent assembly 300 for use in the superior sagittal sinus 130 is constructed of NiTi and includes stent structures and links 305. The inlet zone 310 and transition zone(s) 330/350 of such a stent assembly 300 extends for a nominal length of approximately 5 mm to 25 mm or approximately 10 mm and has a radial force at the inlet zone 310 of approximately 0.0005 N/mm to 0.8 N/mm or approximately 0.6 N/mm increasing through one or more optional transition zone(s) to approximately 0.10 N/mm to 3 N/mm, or approximately 0.8 N/mm at patency zone one 370. The stent structure of the inlet zone 310 and transition zone(s) 330/350 has longitudinal lengths of approximately 0.15 mm to approximately 5 mm or 0.15 mm to approximately 2.3 mm and link 305 lengths of approximately 0.18 mm to approximately 6 mm or 0.18 mm to 2.8 mm and a perimeter of approximately 8 mm to approximately 22 mm. The first patency zone 370 of such a stent assembly 300 can extend for a nominal length of approximately 20 mm to 100 mm or approximately 40 mm and has a radial force of approximately 0.10 N/mm to 3 N/mm, or approximately 1.0 N/mm. The stent structure 372 of the first patency zone 370 can have a length of approximately 0.15 mm to approximately 5 mm or approximately 2.3 mm and link 305 lengths of approximately 0.18 mm to approximately 6 mm or approximately 2.8 mm. The perimeter of the first patency zone 370 can be approximately 9 mm to approximately 22 mm. Various perimeters can be made available to allow for varying patient vessel sizes, such as a series of sizes with perimeters of approximately 16 mm, 19 mm, and 22 mm.

In any of the embodiments described herein, a stent assembly 300 for placement in the superior sagittal sinus 130 through the sigmoid sinus 110 can have one or more patency zones 370, that can be separated by a distance, such as by a transition zone 330, where a first patency zone 370 is located within the superior sagittal sinus 130 and a second patency zone 390 is located within the transverse sinus 120 and can extend into the sigmoid sinus 110.

In some embodiments, an example configuration of the stent assembly 300 for use in the superior sagittal sinus 130 and extending into the sigmoid sinus 110 is as follows: The inlet zone 310 and transition zone(s) 330/350 of such a stent assembly 300 extends for a nominal length of approximately 5 mm to 25 mm and has a radial force at the inlet zone 310 of approximately 0.0005 N/mm to 1.5 N/mm or approximately 0.6 N/mm increasing to approximately 0.10 N/mm to 3 N/mm, or approximately 0.8 N/mm at patency zone one 370. The stent structure of the inlet zone 310 and transition zone(s) 330/350 has longitudinal lengths of approximately 0.15 mm to approximately 5 mm or 0.15 mm to approximately 2.3 mm and link 305 lengths of approximately 0.18 mm to approximately 6 mm or 0.18 mm to 2.8 mm and a perimeter of approximately 8 mm to approximately 28 mm. The first patency zone 370 of such a stent assembly 300 can extend for a nominal length of approximately 20 mm to 100 mm or approximately 45 mm and has a radial force of approximately 0.10 N/mm to 3 N/mm, or approximately 0.8 N/mm. The stent structure 372 of the first patency zone 370 can have a length of approximately 0.15 mm to approximately 5 mm or approximately 2.3 mm and link 305 lengths of approximately 0.15 mm to approximately 6 mm or approximately 2.8 mm. The perimeter of the first patency zone 370 can be approximately 9 mm to approximately 21 mm. The intermediate transition zone 360 can extend for a length of approximately 20 mm to 100 mm or approximately 30 mm and has a radial force of approximately 0.10 N/mm to 3 N/mm, or approximately 0.6 N/mm. A second patency zone 390 can extend for a nominal length of approximately 20 mm to 160 mm or 80 mm and have a radial force of approximately 0.10 N/mm to 3 N/mm, or approximately 0.8 N/mm. Perimeter of the second patency zone 390 can be approximately 14 mm to approximately 26 mm. An optional outlet zone 410 which extends for a length of approximately 3 mm to 15 mm, or 8 mm and has a radial force of 0.05 N/mm to 3 N/mm, or approximately 0.6 N/mm. The perimeter of optional outlet zone 410 of approximately 16 mm to approximately 32 mm. Various perimeters can be made available to allow for varying patient vessel sizes, such as a series of sizes with perimeters of approximately 16 mm, 19 mm, 22 mm, 25 mm and 28 mm.

Embodiments of the stent assembly 300 can be used in procedures for treating dural venous sinus stenoses as described below. Vascular access can be obtained by placing a sheath in the femoral, brachial, cephalic, or jugular vein 100, and a guide catheter and associated guide wire 210 (e.g., 0.035″) can be inserted into the sheath and advanced up to the jugular bulb. The guide catheter may be positioned with the distal end in the jugular bulb or extend inside the sigmoid sinus 110 or beyond. The guide catheter may even be positioned beyond the stenosis. The guide wire 210 and any guide catheter introducer are removed from the patient.

In any of the embodiments described herein, a delivery catheter 200 with a stent assembly 300 is loaded with a guide wire 210 (e.g., 0.014″) and inserted into the guide catheter. The delivery catheter 200 with stent assembly 300 is advanced near the distal end of the guide catheter. The guide wire 210 is advanced until the distal end is beyond the desired location for stent assembly 300 deployment. When the desired location for the inlet end of the stent assembly 300 is adjacent the torcula, the guide wire 210 would typically be advanced either into the superior sagittal sinus 130 or contralateral transverse sinus 120 and down the contralateral jugular vein. The delivery catheter 200 with stent assembly 300 is then advanced over the guide wire 210 until the stent assembly 300 is in the desired location. Optionally, the delivery catheter 200 with stent assembly 300 and guide wire 210 may be moved together or independently in smaller increments through the vasculature. Optionally, the stent assembly 300 can be passed through the stenosis or positioned in the venous sinus system without using a guide wire 210. For positioning the stent assembly 300 to include stenting the superior sagittal sinus 130, the guide wire 210 is advanced into the superior sagittal sinus 130 beyond the target location and the delivery catheter 200 with stent assembly 300 is advanced to the desired location within the superior sagittal sinus 130.

Once the stent assembly 300 is in the desired location, the stent assembly 300 is generally only partially deployed such that a distal portion of the stent assembly 300 engages the vessel wall (e.g., the inlet zone 310 and/or one or more of the transition zones 330/350). The location of the stent assembly 300 may then be evaluated. Optionally, if the stent assembly 300 is not in the desired location, the distal portion of the stent assembly 300 may be recaptured within the delivery catheter 200 and repositioned at another location as previously described. If the stent assembly 300 is at the desired location, deployment is continued until a sufficient length of stent assembly 300 is deployed such that the efficacy implanted device can be assessed. Optionally, if the results are not desirable, the stent assembly 300 may be recaptured and optionally removed from the patient or repositioned. If the results are desirable, the stent assembly 300 is fully deployed.

Optionally, the stent assembly 300 can be loaded in a system that senses intravascular pressure at the proximal and/or distal portion of the target vessel to determine if stent assembly 300 deployment is warranted, or adequate treatment is achieved after or during deployment.

Optionally, the guide wire 210 can be positioned into the superior sagittal sinus 130, contralateral transverse sinus 120, or contralateral jugular vein 100 using a microcatheter. The microcatheter is then removed, and the stent assembly 300 is then loaded onto the wire and tracked into position.

After deploying the stent assembly 300, the delivery catheter 200 and guide wire 210 are retracted into the guide catheter either sequentially or concurrently and removed from the patient. Diagnostics may be conducted to assess placement, physiologic parameters, relief of symptoms, etc., while the guide catheter remains in the patient. The guide catheter is then removed from the patient, followed by the sheath. The access site is then closed.

### EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples may be combined in any combination and placed into a respective independent example. The other examples can be presented in an equivalent manner.

1. A stent assembly for implantation into a blood vessel, comprising:
an inlet zone including at least one inlet stent structure, wherein the inlet stent structure is self-expanding and has an inlet radial force configured to at least partially conform to a non-circular cross-sectional shape of the vessel at least substantially; and
a patency zone having at least one patency stent structure located distal of the inlet stent structure, wherein the patency stent structure is self-expanding and has a patency radial force greater than the inlet radial force, and wherein the patency stent structure is coupled to the inlet stent structure such that the inlet stent structure is configured to be positioned upstream from a narrowing of a lumen along the blood vessel and the patency stent structure is configured to be positioned at the narrowing of the blood vessel lumen.

2. The stent assembly of example 1, and wherein:
the inlet stent structure has at least a generally a circular cross-sectional shape in an unconstrained expanded state and is configured to flex into a generally triangular shape as it expands in contact with the blood vessel; and
the patency stent structure has at least a generally circular cross-sectional shape in an unconstrained expanded state and is configured to flex into a generally triangular shape as it expands in contact with the blood vessel.

3. The stent assembly of any of examples 1-2, wherein:
the inlet radial force of the inlet stent structure is approximately 0.0005 N/mm to 2 N/mm; and
the patency radial force of the patency stent structure is approximately 0.001 N/mm to 3 N/mm.

4. The stent assembly of any of examples 1-3, wherein:
the inlet radial force of the inlet stent structure is approximately 0.001 N/mm to 1.5 N/mm; and
the patency radial force of the patency stent structure is approximately 0.3 N/mm to 2.0 N/mm.

5. The stent assembly of any of examples 1-4, wherein the inlet stent structure has at least a generally circular cross-section shape in an unconstrained expanded state and is configured to flex into a generally triangular cross-sectional shape as it expands in contact with a blood vessel.

6. The stent assembly of any of examples 1-5, wherein the inlet stent structure has at least a generally circular cross-sectional shape in an unconstrained expanded state and is configured to deform into a generally triangular cross-sectional shape as it expands in contact with a generally triangular shaped section of the blood vessel.

7. The stent assembly of any of examples 1-6, wherein the inlet stent structure has at least a generally triangular cross-sectional shape in an unconstrained expanded state and is configured to adapt to the blood vessel as it expands in contact with the blood vessel.

8. The stent assembly of any of examples 1-7, wherein the patency stent structure has at least a generally circular cross-sectional shape in an unconstrained expanded state and a generally circular cross-sectional shape as it expands in contact with the blood vessel.

9. The stent assembly of any of examples 1-8, wherein the patency radial force of the patency stent structure is configured to limit narrowing of the blood vessel when intracranial pressures increase that can cause extrinsic compression or enlargement of arachnoid granulations.

10. The stent assembly of any of examples 1-9, further comprising an outlet zone including at least one outlet stent structure located distal of the patency stent structure, wherein the outlet stent structure is self-expanding and has an outlet radial force less than the patency radial force.

11. The stent assembly of any of examples 1-10, further comprising a transition zone between the inlet zone and the patency zone, wherein the transition zone has a transition stent structure having a transitional radial force greater than the inlet radial force of the inlet stent structure and less than the patency radial force of the patency stent structure.

12. The stent assembly of any of examples 1-11, further comprising a transient pressure zone adjacent to the patency zone and having a transient stent structure with a transient radial force configured to constrict during physiologic spikes in cerebral spinal fluid pressure.

13. The stent assembly of any of examples 1-12 wherein the inlet stent structure comprises an inlet ring having struts and the patency stent structure comprises a patency ring having struts.

14. The stent assembly of any of examples 1-12 wherein the inlet stent structure comprises a plurality of inlet rings having struts and the patency stent structure comprises a plurality of patency rings having struts.

15. The stent assembly of any of examples 1-12 wherein the inlet stent structure comprises a braided mesh and the patency stent structure comprises a patency ring having struts.

16. The stent assembly of any of examples 1-15, further comprising:
an outlet zone including at least one outlet stent structure located distal of the patency stent structure, wherein the outlet stent structure is self-expanding and has an outlet radial force less than the patency radial force;
a transition zone between the inlet zone and the patency zone, wherein the transition zone has a transition stent structure having a transitional radial force greater than the inlet radial force of the inlet stent structure and less than the patency radial force of the patency stent structure; and
a transient pressure zone associated with the patency zone, and the transient zone having a transient stent structure with a transient radial force configured to constrict during physiologic spikes in cerebral spinal fluid pressure.

17. The stent assembly of example 16, wherein:
the transition stent structure is coupled to a distal portion of the inlet stent structure and a proximal portion of the patency stent structure; and the outlet stent structure is coupled to a distal portion of the patency stent structure and a proximal portion of the outlet stent structure.

18. The stent assembly of any of examples 1-17 wherein the patency zone defines a first patency zone having a first patency stent structure and the stent assembly further comprises a second patency zone having a second patency stent structure.

19. The stent assembly of example 18 wherein the first patency stent structure has a first expansion property, and the second patency stent structure has a second expansion property.

20. The stent assembly of example 18 wherein the first patency stent structure has a first patency radial force and the second patency stent structure has a second patency radial force different than the first patency radial force.

21. The stent assembly of example 18, further comprising an intermediate transition zone having an intermediate expansion property different than the first expansion property of the first patency stent structure and the second expansion property of the second patency stent structure such that the intermediate transition stent structure is more flexible than the first and second patency stent structures.

22. The stent assembly of any of examples 1-21 wherein the inlet stent structure and a first patency stent structure are configured to be positioned in the superior sagittal sinus and the second patency stent structure is configured to be positioned at least in part in the transverse sinus, and wherein the stent assembly further comprises a transition zone having a transition stent structure with a lower radial force than the first or second patency stent structures.

23. The stent assembly of any of examples 1-22 wherein the patency zone includes at least one patency stent structure that is self-expanding and has a patency radial force configured to engage arachnoid granulations within a lumen of the blood vessel and expand to restore flow within the lumen of the blood vessel.

24. A stent assembly for implantation into a blood vessel, comprising:
an inlet zone including at least one inlet stent structure, wherein the inlet stent structure is self-expanding and has an inlet radial force that compresses with increases in external pressure on the blood vessel; and
a patency zone having at least one patency stent structure, wherein the patency stent structure is self-expanding and has a patency radial force greater than the inlet radial force, and the patency radial force being sufficient to maintain patency of the blood vessel when external pressure on the blood vessel increases.

25. The stent assembly of example 24, and wherein:
the inlet stent structure has at least a generally a circular cross-sectional shape in an unconstrained expanded state and is configured to flex into a generally triangular shape as it expands in contact with the blood vessel; and the patency stent structure has at least a generally circular cross-sectional shape in an unconstrained expanded state and is configured to flex into a generally triangular shape as it expands in contact with the blood vessel.

26. The stent assembly of any of examples 24-25 wherein:
the inlet stent structure has at least a generally a circular cross-sectional shape in an unconstrained expanded state and is configured to flex into a generally triangular shape as it expands in contact with the blood vessel; and
the patency stent structure has at least a generally circular cross-sectional shape in an unconstrained expanded state and a generally circular cross-sectional shape as it expands in contact with the blood vessel, whereby a portion of the blood vessel in contact with the patency stent structure at least substantially conforms to the generally circular cross-sectional shape of the patency stent structure.

27. The stent assembly of any of examples 24-26, wherein:
the inlet radial force of the inlet stent structure is approximately 0.0005 N/mm to 2 N/mm; and
the patency radial force of the patency stent structure is approximately 0.001 N/mm to 3 N/mm.

28. The stent assembly of any of examples 24-27, wherein the inlet stent structure has at least a generally circular cross-section shape in an unconstrained expanded state and is configured to flex into a generally triangular cross-sectional shape as it expands in contact with a blood vessel.

29. The stent assembly of any of examples 24-28, wherein the inlet stent structure has at least a generally circular cross-sectional shape in an unconstrained expanded state and is configured to deform into a generally triangular cross-sectional shape as it expands in contact with a generally triangular shaped section of the blood vessel.

30. The stent assembly of any of examples 24-29, wherein the inlet stent structure has at least a generally triangular cross-sectional shape in an unconstrained expanded state and is configured to adapt to the blood vessel as it expands in contact with the blood vessel.

31. The stent assembly of any of examples 24-29, wherein the patency radial force of the patency stent structure is configured to limit constriction of the blood vessel when external pressure on the blood vessel increases.

32. The stent assembly of any of examples 24-31, further comprising an outlet zone including at least one outlet stent structure located distal of the patency stent structure, wherein the outlet stent structure is self-expanding and has an outlet radial force less than the patency radial force.

33. The stent assembly of any of examples 24-32, further comprising a transition zone between the inlet zone and the patency zone, wherein the transition zone has a transition stent structure having a transitional radial force greater than the inlet radial force of the inlet stent structure and less than the patency radial force of the patency stent structure.

34. The stent assembly of any of examples 24-33 further comprising a transient pressure zone adjacent to the patency zone and having a transient stent structure with a transient radial force configured to constrict during physiologic spikes in cerebral spinal fluid pressure.

35. The stent assembly of any of examples 24-34 wherein the inlet stent structure comprises an inlet ring having struts and the patency stent structure comprises a patency ring having struts.

36. The stent assembly of any of examples 24-35 wherein the inlet stent structure comprises a plurality of inlet rings having struts and the patency stent structure comprises a plurality of patency rings having struts.

37. The stent assembly of any of examples 24-36 wherein the inlet stent structure comprises a braided mesh and the patency stent structure comprises a patency ring having struts.

38. The stent assembly of any of examples 24-37, further comprising:
an outlet zone including at least one outlet stent structure located distal of the patency stent structure, wherein the outlet stent structure is self-expanding and has an outlet radial force less than the patency radial force;
a transition zone between the inlet zone and the patency zone, wherein the transition zone has a transition stent structure having a transitional radial force greater than the inlet radial force of the inlet stent structure and less than the patency radial force of the patency stent structure; and
a transient pressure zone between the patency zone and the outlet zone, and the transient zone having a transient stent structure with a transient radial force configured to constrict during physiologic spikes in cerebral spinal fluid pressure.

39. The stent assembly of any of examples 24-38, wherein:
the transition stent structure is coupled to a distal portion of the inlet stent structure and a proximal portion of the patency stent structure; and
the outlet stent structure is coupled to a distal portion of the patency stent structure and a proximal portion of the outlet stent structure.

40. The stent assembly of any of examples 24-39 wherein the patency zone defines a first patency zone having a first patency stent structure and the stent assembly further comprises a second patency zone having a second patency stent structure.

41. The stent assembly of any of examples 24-40 wherein the first patency stent structure has a first expansion property, and the second patency stent structure has a second expansion property.

42. The stent assembly of example 40 wherein the first patency stent structure has a first patency radial force and the second patency stent structure has a second patency radial force different than the first patency radial force.

43. The stent assembly of example 40, further comprising an intermediate transition zone having an intermediate expansion property different than the first expansion property of the first patency stent structure and the second expansion property of the second patency stent structure such that the intermediate transition stent structure is more flexible than the first and second patency stent structures.

44. A stent assembly for implantation into a blood vessel, comprising:
an inlet zone including at least one inlet stent structure, wherein the inlet stent structure is self-expanding and is configured to at least substantially conform to a non-circular cross-sectional shape of a portion of the blood vessel in contact with the inlet stent structure as the inlet stent structure expands; and
a patency zone having at least one patency stent structure, wherein the patency stent structure is self-expanding and is configured to expand to an expanded shape different than the non-circular cross-sectional shape of the vessel whereby a portion of the vessel in contact with the patency stent structure at least substantially conforms to the expanded shape of the patency stent structure, and the patency stent structure is coupled to the inlet stent structure such that the inlet stent structure is configured to be positioned upstream from a narrowing along the blood vessel and the patency stent structure is configured to be positioned at the point of luminal compromise.

45. The stent assembly of example 44, wherein:
the inlet stent structure has an inlet radial force; and
the patency stent structure has a patency radial force greater than the inlet radial force.

46. The stent assembly of any of examples 44-45, wherein:
the inlet stent structure has at least a generally a circular cross-sectional shape in an unconstrained expanded state and is configured to flex into a generally triangular shape as it expands in contact with the blood vessel; and
the patency stent structure has at least a generally circular cross-sectional shape in an unconstrained expanded state and is configured to flex into a generally triangular shape as it expands in contact with the blood vessel.

47. The stent assembly of any of examples 44-46, wherein:
the inlet radial force of the inlet stent structure is approximately 0.0005 N/mm to 2 N/mm; and
the patency radial force of the patency stent structure is approximately 0.001 N/mm to 3 N/mm.

48. The stent assembly of any of examples 44-47, wherein the inlet stent structure has at least a generally circular cross-section shape in an unconstrained expanded state and is configured to flex into a generally triangular cross-sectional shape as it expands in contact with a blood vessel.

49. The stent assembly of any of examples 44-48, wherein the inlet stent structure has at least a generally circular cross-sectional shape in an unconstrained expanded state and is configured to deform into a generally triangular cross-sectional shape as it expands in contact with a generally triangular shaped section of the blood vessel.

50. The stent assembly of any of examples 44-49, wherein the inlet stent structure has at least a generally triangular cross-sectional shape in an unconstrained expanded state and is configured to adapt to the blood vessel as it expands in contact with the blood vessel.

51. The stent assembly of any of examples 44-50, wherein the patency stent structure has at least a generally circular cross-sectional shape in an unconstrained expanded state and a generally circular cross-sectional shape as it expands in contact with the blood vessel.

52. The stent assembly of any of examples 44-51, wherein the patency radial force of the patency stent structure is configured to limit constriction of the blood vessel when external pressure on the blood vessel increases.

53. The stent assembly of any of examples 44-52, further comprising an outlet zone including at least one outlet stent structure located distal of the patency stent structure, wherein the outlet stent structure is self-expanding and has an outlet radial force less than the patency radial force.

54. The stent assembly of any of examples 44-53, further comprising a transition zone between the inlet zone and the patency zone, wherein the transition zone has a transition stent structure having a transitional radial force greater than the inlet radial force of the inlet stent structure and less than the patency radial force of the patency stent structure.

55. The stent assembly of any of examples 44-54, further comprising a transient pressure zone adjacent to the patency zone and having a transient stent structure with a transient radial force configured to constrict during physiologic spikes in cerebral spinal fluid pressure.

56. The stent assembly of any of examples 44-55 wherein the inlet stent structure comprises an inlet ring having struts and the patency stent structure comprises a patency ring having struts.

57. The stent assembly of any of examples 44-56 wherein the inlet stent structure comprises a plurality of inlet rings having struts and the patency stent structure comprises a plurality of patency rings having struts.

58. The stent assembly of any of examples 44-57 wherein the inlet stent structure comprises a braided mesh and the patency stent structure comprises a patency ring having struts.

59. The stent assembly of any of examples 44-58, further comprising:
an outlet zone including at least one outlet stent structure located distal of the patency stent structure, wherein the outlet stent structure is self-expanding and has an outlet radial force less than the patency radial force;
a transition zone between the inlet zone and the patency zone, wherein the transition zone has a transition stent structure having a transitional radial force greater than the inlet radial force of the inlet stent structure and less than the patency radial force of the patency stent structure; and
a transient pressure zone associated with the patency zone, and the transient zone having a transient stent structure with a transient radial force configured to constrict during physiologic spikes in cerebral spinal fluid pressure.

60. The stent assembly of any of examples 44-59, wherein:
the transition stent structure is coupled to a distal portion of the inlet stent structure and a proximal portion of the patency stent structure; and
the outlet stent structure is coupled to a distal portion of the patency stent structure.

61. The stent assembly of any of examples 44-60 wherein the patency zone defines a first patency zone having a first patency stent structure and the stent assembly further comprises a second patency zone having a second patency stent structure.

62. The stent assembly of any of examples 44-61 wherein the first patency stent structure has a first expansion property, and the second patency stent structure has a second expansion property.

63. The stent assembly of example 61 wherein the first patency stent structure has a first patency radial force and the second patency stent structure has a second patency radial force different than the first patency radial force.

64. The stent assembly of example 61, further comprising an intermediate transition zone having an intermediate expansion property different than the first expansion property of the first patency stent structure and the second expansion property of the second patency stent structure such that the intermediate transition stent structure is more flexible than the first and second patency stent structures.

65. The stent assembly of any of examples 44-63 wherein the inlet stent structure and a first patency stent structure are configured to be positioned in the superior sagittal sinus and the second patency stent structure is configured to be positioned at least in part in the transverse sinus, and wherein the stent assembly further comprises a transition zone having a transition stent structure with a lower radial force than the first or second patency stent structures.

66. A method of treating an indication caused by a narrowing along a dural venous sinus, comprising:
positioning a stent assembly in a dural venous sinus such that (a) an inlet zone of the stent assembly is at an upstream location from the point of luminal compromise in the dural venous sinus relative to blood flow through the dural venous sinus and (b) a patency zone of the stent assembly is at the narrowed portion of the dural venous sinus;
expanding an inlet stent structure of the inlet zone to have a triangular cross-section shape that at least substantially approximate a generally triangular cross-sectional shape of the dural venous sinus at the upstream location, wherein the inlet stent structure has an inlet radial force; and
expanding a patency stent structure of the patency zone at the narrowed portion of the dural venous sinus such that the patency stent structure increases a cross-sectional area of the narrowed portion, wherein the patency stent structure has a patency radial force greater than the inlet radial force.

67. The method of example 66 wherein the inlet stent structure has a circular cross-sectional shape in an expanded unconstrained state and expanding the inlet stent structure comprises self-expanding the inlet stent structure such that it at least substantially approximates the triangular cross-section shape of the upstream location.

68. The method of any of any of examples 66-67 wherein the inlet stent structure is coupled to the patency stent structure before positioning the stent assembly in the dural venous sinus such that a single stent assembly is positioned along the upstream location and the narrowed portion of the dural venous sinus.

69. The method of any of examples 66-68 wherein the indication is papilledema.

70. The method of any of examples 66-68 wherein the indication is pulsatile tinnitus.

71. The method of any of examples 66-68 wherein the indication is headaches.

72. The method of any of examples 66-71 wherein:
the inlet stent structure has a circular cross-sectional shape in an expanded unconstrained state and expanding the inlet stent structure comprises self-expanding the inlet stent structure such that it at least substantially approximates the triangular cross-sectional shape of the upstream location; and
the patency stent structure has a circular cross-sectional shape in an expanded unconstrained state and expanding the patency stent structure comprises self-expanding the patency stent structure such that it at least substantially approximates a triangular cross-sectional shape of the narrowed portion of the dural venous sinus.

73. The method of example 72 wherein, upon expansion, the triangular cross-sectional shape of the inlet stent structure is different than the triangular cross-sectional shape of the patency stent structure.

74. The method of any of examples 66-73 wherein positioning the stent assembly in the dural venous sinus further comprises locating an outlet zone of the stent assembly at a downstream location from the narrowed portion of the dural venous sinus relative to blood flow through the dural venous sinus, and wherein the outlet zone has an outlet stent structure with an outlet radial expansion force greater less than the patency expansion force.

75. The method of example 74, further comprising expanding an outlet stent structure of the outlet zone at the downstream location such that the outlet stent structure has a cross-sectional dimension approximating a cross-sectional dimension of the downstream location.

Alternate embodiments may be devised without departing from the spirit or the scope of the present technology. Additionally, well-known elements of embodiments of the systems, apparatuses, and methods have not been described in detail or have been omitted so as not to obscure the relevant details of the systems, apparatuses, and methods.

The terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

When the terms “coupled” and “connected,” along with their derivatives, are used, these terms are not intended as synonyms for each other. For example, “connected” may be used to indicate that two or more elements are in direct physical or electrical contact with each other. “Coupled” may mean that two or more elements are in direct physical or electrical contact (e.g., directly coupled) or that two or more elements are not in direct contact with each other but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Various embodiments of the systems, apparatuses, and methods have been described, and in many of the different embodiments many features are similar. To avoid redundancy, repetitive description of these similar features may not be made in some circumstances. It shall be understood, however, that description of a first-appearing feature applies to the later described similar feature and each respective description, therefore, is to be incorporated therein without such repetition.

From the foregoing, it will be appreciated that specific embodiments of the disclosure have been described herein for purposes of illustration, but that various modifications may be made without deviating from the scope of the disclosure. Accordingly, the disclosure is not limited except as by the appended claims.

I/We claim:

1. A stent assembly for implantation into a blood vessel, comprising:
an inlet zone including at least one inlet stent structure, wherein the inlet stent structure is self-expanding and has an inlet radial force configured to at least partially conform to a non-circular cross-sectional shape of the vessel; and
a patency zone having at least one patency stent structure located distal of the inlet stent structure, wherein the patency stent structure is self-expanding and has a patency radial force greater than the inlet radial force, and wherein the patency stent structure is coupled to the inlet stent structure such that the inlet stent structure is configured to be positioned upstream from a narrowing of a lumen along the blood vessel and the patency stent structure is configured to be positioned at the narrowing of the blood vessel lumen.

2. The stent assembly of claim 1, and wherein:
the inlet stent structure has at least a generally a circular cross-sectional shape in an unconstrained expanded state and is configured to flex into a generally triangular shape as it expands in contact with the blood vessel; and
the patency stent structure has at least a generally circular cross-sectional shape in an unconstrained expanded state and is configured to flex into a generally triangular shape as it expands in contact with the blood vessel.

3. The stent assembly of claim 1, wherein:
the inlet radial force of the inlet stent structure is approximately 0.0005 N/mm to 2 N/mm; and
the patency radial force of the patency stent structure is approximately 0.001 N/mm to 3 N/mm.

4. The stent assembly of claim 1, wherein:
the inlet radial force of the inlet stent structure is approximately 0.001 N/mm to 1.5 N/mm; and
the patency radial force of the patency stent structure is approximately 0.3 N/mm to 2.0 N/mm.

5. The stent assembly of claim 1, wherein the inlet stent structure has at least a generally circular cross-section shape in an unconstrained expanded state and is configured to flex into a generally triangular cross-sectional shape as it expands in contact with a blood vessel.

6. The stent assembly of claim 1, wherein the inlet stent structure has at least a generally circular cross-sectional shape in an unconstrained expanded state and is configured to deform into a generally triangular cross-sectional shape as it expands in contact with a generally triangular shaped section of the blood vessel.

7. The stent assembly of claim 1, wherein the inlet stent structure has at least a generally triangular cross-sectional shape in an unconstrained expanded state and is configured to adapt to the blood vessel as it expands in contact with the blood vessel.

8. The stent assembly of claim 1, wherein the patency stent structure has at least a generally circular cross-sectional shape in an unconstrained expanded state and a generally circular cross-sectional shape as it expands in contact with the blood vessel.

9. The stent assembly of claim 1, wherein the patency radial force of the patency stent structure is configured to limit narrowing of the blood vessel when intracranial pressures increase that can cause extrinsic compression or enlargement of arachnoid granulations.

10. The stent assembly of claim 1, further comprising an outlet zone including at least one outlet stent structure located distal of the patency stent structure, wherein the outlet stent structure is self-expanding and has an outlet radial force less than the patency radial force.

11. The stent assembly of claim 1, further comprising a transition zone between the inlet zone and the patency zone, wherein the transition zone has a transition stent structure having a transitional radial force greater than the inlet radial force of the inlet stent structure and less than the patency radial force of the patency stent structure.

12. The stent assembly of claim 1, further comprising a transient pressure zone adjacent to the patency zone and having a transient stent structure with a transient radial force configured to constrict during physiologic spikes in cerebral spinal fluid pressure.

13. The stent assembly of claim 1 wherein the inlet stent structure comprises an inlet ring having struts and the patency stent structure comprises a patency ring having struts.

14. The stent assembly of claim 1 wherein the inlet stent structure comprises a plurality of inlet rings having struts and the patency stent structure comprises a plurality of patency rings having struts.

15. The stent assembly of claim 1 wherein the inlet stent structure comprises a braided mesh and the patency stent structure comprises a patency ring having struts.

16. The stent assembly of claim 1, further comprising:
an outlet zone including at least one outlet stent structure located distal of the patency stent structure, wherein the outlet stent structure is self-expanding and has an outlet radial force less than the patency radial force;
a transition zone between the inlet zone and the patency zone, wherein the transition zone has a transition stent structure having a transitional radial force greater than the inlet radial force of the inlet stent structure and less than the patency radial force of the patency stent structure; and
a transient pressure zone associated with the patency zone, and the transient pressure zone having a transient stent structure with a transient radial force configured to constrict during physiologic spikes in cerebral spinal fluid pressure.

17. The stent assembly of claim 16, wherein:
the transition stent structure is coupled to a distal portion of the inlet stent structure and a proximal portion of the patency stent structure; and
the outlet stent structure is coupled to a distal portion of the patency stent structure and a proximal portion of the outlet stent structure.

18. The stent assembly of claim 1 wherein the patency zone defines a first patency zone having a first patency stent structure and the stent assembly further comprises a second patency zone having a second patency stent structure.

19. The stent assembly of claim 18 wherein the first patency stent structure has a first expansion property, and the second patency stent structure has a second expansion property.

20. The stent assembly of claim 18 wherein the first patency stent structure has a first patency radial force and the second patency stent structure has a second patency radial force different than the first patency radial force.

21. The stent assembly of claim 19, further comprising an intermediate transition zone having an intermediate expansion property different than the first expansion property of the first patency stent structure and the second expansion property of the second patency stent structure such that the intermediate transition zone is more flexible than the first and second patency stent structures.

22. The stent assembly of claim 18 wherein the inlet stent structure and the first patency stent structure are configured to be positioned in the superior sagittal sinus and the second patency stent structure is configured to be positioned at least in part in the transverse sinus, and wherein the stent assembly further comprises a transition zone having a transition stent structure with a lower radial force than the first or second patency stent structures.

23. The stent assembly of any claim 1 wherein the patency zone includes at least one patency stent structure that is self-expanding and has a patency radial force configured to engage arachnoid granulations within a lumen of the blood vessel and expand to restore flow within the lumen of the blood vessel.

24. A method of treating an indication caused by a narrowing along a dural venous sinus, comprising:
positioning a stent assembly in a dural venous sinus such that (a) an inlet zone of the stent assembly is at an upstream location from a point of luminal compromise in the dural venous sinus relative to blood flow through the dural venous sinus and (b) a patency zone of the stent assembly is at the narrowed portion of the dural venous sinus;
expanding an inlet stent structure of the inlet zone to have a triangular cross-section shape that at least substantially approximate a generally triangular cross-sectional shape of the dural venous sinus at the upstream location, wherein the inlet stent structure has an inlet radial force; and
expanding a patency stent structure of the patency zone at the narrowed portion of the dural venous sinus such that the patency stent structure increases a cross-sectional area of the narrowed portion, wherein the patency stent structure has a patency radial force greater than the inlet radial force.

25. The method of claim 24 wherein the inlet stent structure has a circular cross-sectional shape in an expanded unconstrained state and expanding the inlet stent structure comprises self-expanding the inlet stent structure such that it at least substantially approximates the triangular cross-section shape of the upstream location.

26. The method of claim 24 wherein the inlet stent structure is coupled to the patency stent structure before positioning the stent assembly in the dural venous sinus such that a single stent assembly is positioned along the upstream location and the narrowed portion of the dural venous sinus.

27. The method of claim 24 wherein the indication is papilledema.

28. The method of claim 24 wherein the indication is pulsatile tinnitus.

29. The method of claim 24 wherein the indication is headaches.

30. The method of claim 24 wherein:
the inlet stent structure has a circular cross-sectional shape in an expanded unconstrained state and expanding the inlet stent structure comprises self-expanding the inlet stent structure such that it at least substantially approximates the triangular cross-sectional shape of the upstream location; and
the patency stent structure has a circular cross-sectional shape in an expanded unconstrained state and expanding the patency stent structure comprises self-expanding the patency stent structure such that it at least substantially approximates a triangular cross-sectional shape of the narrowed portion of the dural venous sinus.

31. The method of claim 30 wherein, upon expansion, the triangular cross-sectional shape of the inlet stent structure is different than the triangular cross-sectional shape of the patency stent structure.

32. The method of claim 30 wherein positioning the stent assembly in the dural venous sinus further comprises locating an outlet zone of the stent assembly at a downstream location from the narrowed portion of the dural venous sinus relative to blood flow through the dural venous sinus, and wherein the outlet zone has an outlet stent structure with an outlet radial expansion force greater less than the patency expansion force.

33. The method of claim 32, further comprising expanding an outlet stent structure of the outlet zone at the downstream location such that the outlet stent structure has a cross-sectional dimension approximating a cross-sectional dimension of the downstream location.
